(19) United States
(12) Patent Application Publication
(10) Pub. No.: US 2026/0071271 A1
(43) Pub. Date: Mar. 12, 2026

(54) FLOW CELL BONDING MATERIALS

(71) Applicant: ILLUMINA, INC., San Diego, CA (US)

(72) Inventors: Anurag Agrawal, San Diego, CA (US); Allison Duchnak, Escondido, CA (US); Andrew Enslow, Escondido, CA (US); Casey Glick, Belmont, CA (US); Tae Joon Kwak, San Diego, CA (US); Steven Modiano, San Diego, CA (US); Rean Silke Musa, San Diego, CA (US); Michael Rapp, La Jolla, CA (US); Gordon Ringwald, Carlsbad, CA (US); Yekaterina Rokhlenko, San Diego, CA (US); Hua Wang, San Diego, CA (US); Adam White, San Diego, CA (US)

(21) Appl. No.: 19/320,685

(22) Filed: Sep. 5, 2025

Related U.S. Application Data
(60) Provisional application No. 63/693,879, filed on Sep. 12, 2024.

(51) Int. Cl. C12Q 1/6874; B01L 3/00

(52) U.S. Cl. CPC C12Q 1/6874; B01L 3/502707; B01L 3/502761; B01L 2200/0647; B01L 2300/069; B01L 2300/12

(57) ABSTRACT

An example of a flow cell includes a first substrate having an active region and a bonding region that is spatially separate from the active region. A polymeric hydrogel is positioned at the active region. The flow cell further includes an interposer having a first side that is attached to the bonding region and having a second side that is opposed to the first side. A light-switchable bonding material is in contact with at least one of the first side or the second side, and a lid or a second substrate is attached to the second side of the interposer.

### CROSS-REFERENCE TO RELATED APPLICATION

This application claims the benefit of U.S. Provisional Application Ser. No. 63/693,879, filed Sep. 12, 2024, the content of which is incorporated by reference herein in its entirety.

### REFERENCE TO SEQUENCE LISTING

The instant application contains a Sequence Listing which has been submitted electronically in XML file format and is hereby incorporated by reference in its entirety. Said XML copy, created on Sep. 3, 2025 is named IL1281B_IP-2775-US_Sequence_Listing.xml and is 17,767 bytes in size.

### BACKGROUND

Various protocols in biological or chemical research involve performing a large number of controlled reactions on local support surfaces or within predefined reaction chambers, or active regions. The reactions may then be observed or detected, and subsequent analysis may help identify or reveal properties of chemicals involved in the controlled reactions. In some examples, the reactions generate fluorescence, and thus an optical system that is configured for fluorescence detection may be used to analyze the controlled reactions.

### SUMMARY

Disclosed herein are flow cells and methods utilizing bonding materials that enable on-demand attachment and/or separation of flow cell components. In some instances, the bonding materials are chemically modifiable (i.e., chemically “switchable”). The switchable bonding materials may be used to attach and/or detach various flow cell components (e.g., flow cell substrates, interposers, lids, etc.) to/from one another at desired times. In some examples disclosed herein, the switchable bonding materials are structurally modified upon exposure to preselected light wavelengths, and this structural modification facilitates the attachment or detachment of flow cell components having the switchable bonding materials in contact therewith. In other examples disclosed herein, the switchable bonding materials are structurally modified during a timed exposure to a preselected solvent, and this modification facilitates the detachment of flow cell components having the switchable bonding material(s) in contact therewith. In still other examples disclosed herein, the switchable bonding materials are structurally modified during a timed exposure to a predetermined temperature (e.g., heat), and this modification facilitates the detachment of flow cell components having the switchable bonding material(s) in contact therewith. In yet other examples disclosed herein, the bonding material(s) are non-switchable, but can still facilitate the on-demand attachment and/or separation of flow cell components.

The ability to selectively attach and/or detach flow cell components to/from one another using the bonding materials disclosed herein facilitates versatility in both flow cell component configurations and flow cell uses. Further, the bonding materials disclosed herein allow individual flow cell components to be attached or detached to/from one another on-demand while leaving behind minimal or no amount(s) of bonding material residue, which can aid in reusability of the flow cell components.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of an example of a flow cell disclosed herein;

FIG. 1B is an enlarged, perspective and partially-cutaway view of an example of a flow cell including a structure having a lane defined therein, the lane defining an active region;

FIG. 1C is an enlarged, perspective and partially-cutaway view of another example of a flow cell including a structure that is patterned with a plurality of functionalized pads included in an active region;

FIG. 2A is a cross-sectional view, taken along line 2A-2A of FIG. 1A, of an example of a flow cell including a first substrate bonded to a lid, where the first substrate includes a plurality of depressions defined in discrete active regions;

FIG. 2B is a cross-sectional view, taken along line 2B-2B of FIG. 1A, of another example of a flow cell including a first substrate bonded to a lid, where the first substrate includes i) a plurality of depressions defined in discrete active regions, and ii) a plurality of depressions defined in discrete bonding regions;

FIG. 2C is a cross-sectional view, taken along line 2C-2C of FIG. 1A, of another example of a flow cell including a first substrate bonded to a second substrate, where the first substrate and the second substrate respectively include i) a plurality of depressions defined in discrete active regions, and ii) a plurality of depressions defined in discrete bonding regions;

FIG. 2D is a cross-sectional view, taken along line 2D-2D of FIG. 1A, of another example of a flow cell including a first substrate bonded to a second substrate, where the first and second substrates respectively include a plurality of depressions defined in discrete active regions, and where the first substrate also includes a plurality of depressions defined in discrete bonding regions;

FIG. 3A through FIG. 3C is a schematic view of examples of a method of forming a flow cell that includes a switchable bonding material, where FIG. 3A depicts a substrate having a lane defined therein, FIG. 3B depicts attaching a first side of an interposer to (a) bonding region(s) of the substrate using a bonding material, and FIG. 3C depicts attaching a lid or a second substrate to a second side of the interposer using a bonding material;

FIG. 4A through FIG. 4D is a schematic view of examples of a method of using a flow cell that includes a switchable bonding material, where FIG. 4A depicts a flow cell including a first substrate bonded to a first side of an interposer and a lid or a second substrate bonded to a second side of the interposer, FIG. 4B depicts detaching the interposer (and the lid or the second substrate attached thereto) from the first substrate, FIG. 4C depicts detaching the lid or the second substrate from the interposer, where the interposer remains attached to the first substrate, and FIG. 4D depicts (i) removing the interposer from the first substrate and (ii) removing the lid or the second substrate from the interposer;

FIG. 5 depicts the peel strength (in newtons/mm) for twelve example flow cells, each of which included a light-switchable bonding material, that were exposed to a peel strength test either without UV exposure (0 seconds) or after UV exposure (20 seconds);

FIG. 6 is a graphical representation of the results of shearing torque tests that were performed on eight example flow cells, each of which included a solvent-responsive bonding material, after dry periods and after different time periods of soaking in tap water or deionized water, with the tested flow cell groups being shown on the x axis and shearing force (in Ib*in) being shown on the y axis;

FIG. 7A is a black-and-white reproduction of an optical microscope image obtained from a flow cell, which included a solvent-responsive bonding material, after a de-bonding operation that took place without any solvent soak;

FIG. 7B is a black-and-white reproduction of an optical microscope image obtained from a flow cell, which included a solvent-responsive bonding material, after a de-bonding operation that took place after the flow cell was exposed to soaking; and

FIG. 8A and FIG. 8B are graphs that collectively depict a statistical correlation of the predicted spatial location of sequenced DNA samples on a flow cell substrate, providing favorable indicia that a debonding process disclosed herein did not deleteriously interfere with biological sequencing.

### DETAILED DESCRIPTION

Flow cells used in nucleic acid sequencing may include one or more substrates having (an) active region(s) where amplification, cluster generation, and sequencing can take place. These flow cells include a substrate that is bonded to a lid or to another substrate, e.g., at one or more bonding region(s), to create an enclosed space for delivering reagents to the active region(s). It may be desirable for the substrate to be bonded to the lid or to the other substrate using a switchable bonding material, such that the substrate can be bonded to (or de-bonded from) the lid or the second substrate upon exposure to a preselected reaction condition. Further, to promote material reusability, it may be desirable for the bonding/de-bonding of the substrate to/from the lid (or to/from the second substrate) to be performed such that minimal or no amount(s) of residue (e.g., of the switchable bonding material) is/are left behind after de-bonding is performed.

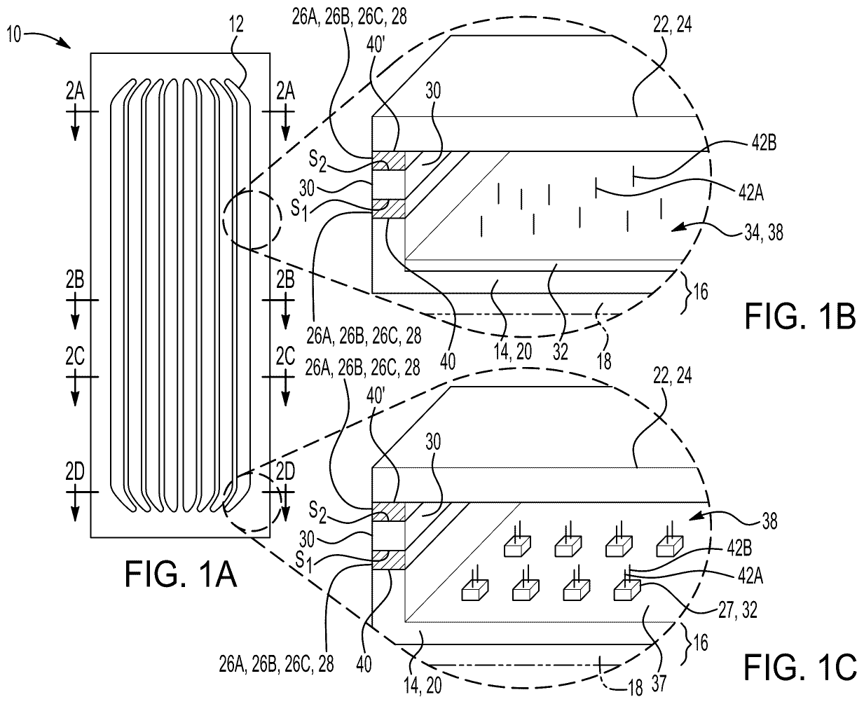

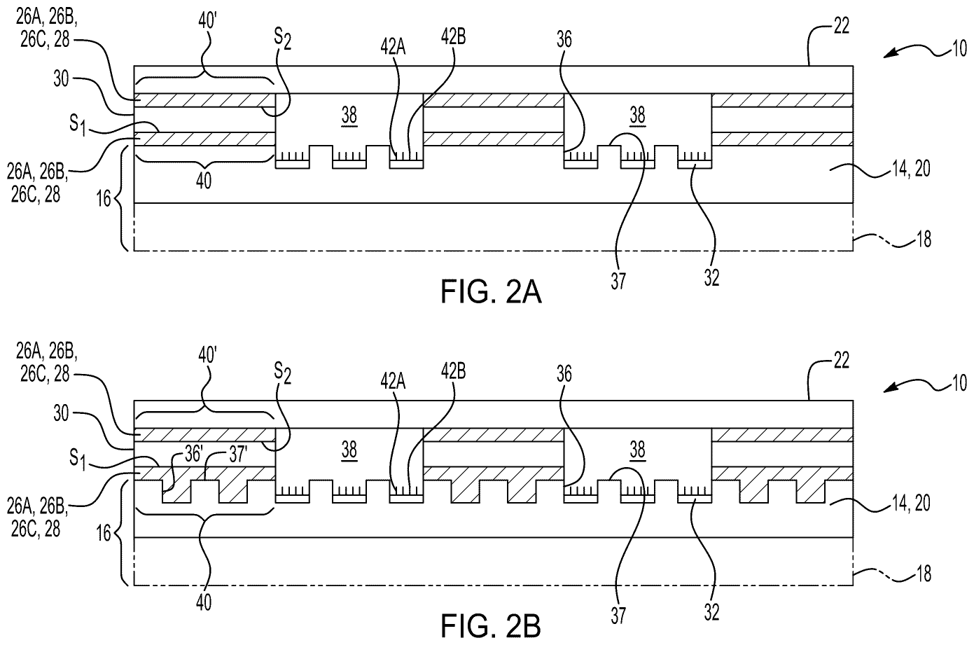

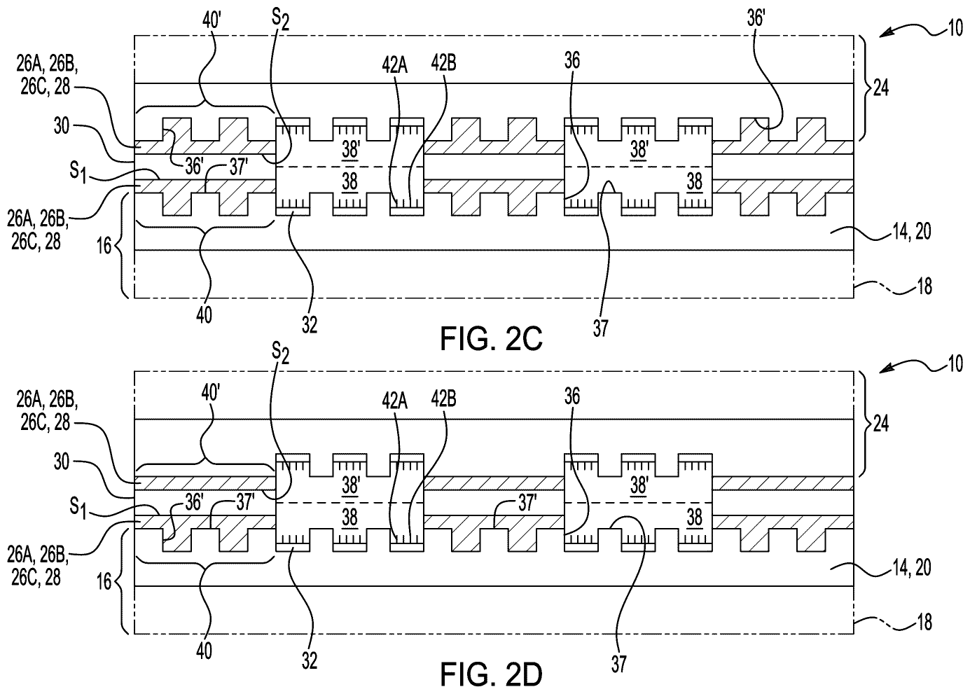

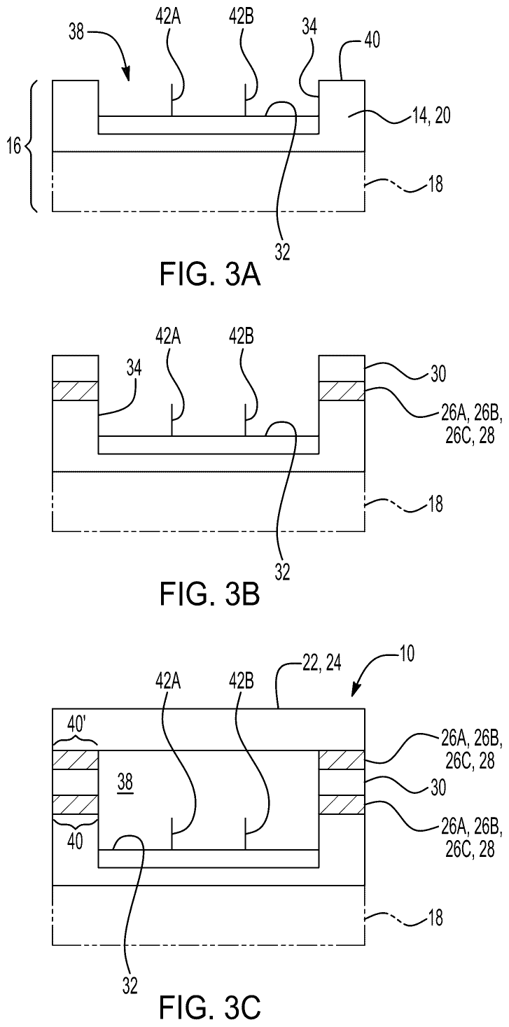

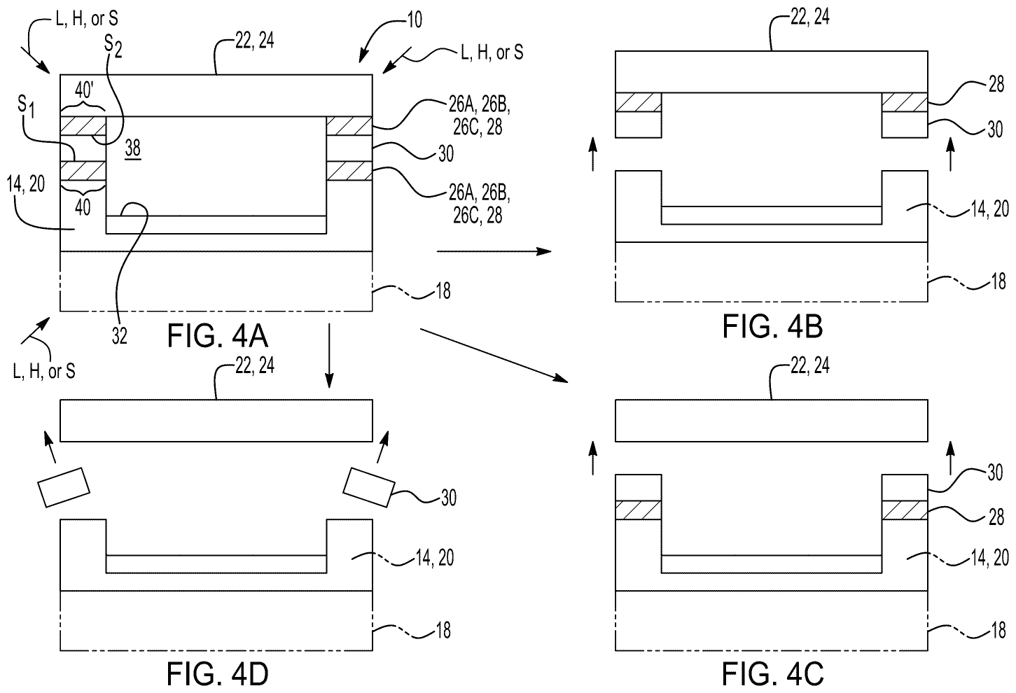

Disclosed herein are flow cells including switchable and/or non-switchable bonding materials that facilitate on-demand bonding and de-bonding of the components included in the flow cell. The structure of the flow cell and methods of forming some aspects thereof will be described in reference to FIG. 1A through FIG. 1C and in reference to FIG. 2A through FIG. 2D. Examples of a method of forming the flow cell will be described in reference to FIG. 3A through FIG. 3C. Examples of a method of using the flow cell will be described in reference to FIG. 4A through FIG. 4D.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, adjacent, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

The term “acrylamide monomer,” as used herein, refers to a monomer with the structure

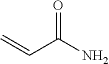

or a monomer including an acrylamide group. Examples of the monomer including an acrylamide group include azido acetamido pentyl acrylamide:

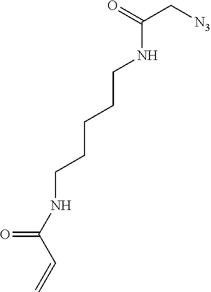 [SMILES: C=CC(=O)NCCCCCNC(=O)CN=[N+]=[N-]]

### and N-isopropylacrylamide:

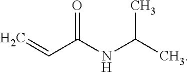

Other acrylamide monomers may be used.

The term “activation,” as used herein, refers to a process that generates reactive groups at the surface of a single layer substrate or an outermost layer of a multi-layered substrate. Activation may be accomplished using silanization or plasma ashing. Though not explicitly shown in the figures, when activation of a surface is performed, it is to be understood that silane groups or —OH (e.g., hydroxy or hydroxyl) functional groups become introduced to the surface. These functional groups can then be used to covalently attach a material, such as a polymeric hydrogel, to the activated surface that includes the functional groups.

The term “active region,” as used herein, refers to a region of a substrate where a reaction can be carried out. The active regions include a polymeric hydrogel that has primers attached thereto (or that is capable of attaching primers thereto), which may be referred to as “surface chemistry.” One or more active region(s) may be in fluid communication with a flow channel. The term “active region” may refer to a lane that is defined in a substrate (see the lane 34 in FIG. 1B), or an area of a substrate having functionalized pads thereon (see the functionalized pads 27 in the active region 38 in FIG. 1C), or an area of a substrate including depressions defined therein, where the depressions include surface chemistry (see the depressions 36 in the active region(s) 38 in each of FIG. 2A through FIG. 2D). In the example flow cells disclosed herein that include two substrates attached to one another, each of the two substrates may include (a) respective active region(s) (see, e.g., FIG. 2C and FIG. 2D, each of which shows a first substrate 14, 16 including (first) active regions 38 and a second substrate 24 including (second) active regions 38′).

The term “aldehyde,” as used herein, refers to an organic compound containing a functional group with the structure —CHO, which includes a carbonyl center (i.e., a carbon double-bonded to oxygen) with the carbon atom also bonded to hydrogen and an R group, such as an alkyl or other side chain. The general structure of an aldehyde is:

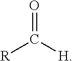

The term “alkyl,” as used herein, refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and tert-butyl.

The term “alkenyl,” as used herein, refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

The terms “alkyne” or “alkynyl,” as used herein, refer to a straight or branched hydrocarbon chain containing one or more triple bonds. The alkynyl group may have 2 to 20 carbon atoms.

The term “aryl,” as used herein, refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amino” functional group refers to an —NRₐR_{b} group, where Rₐ and R_{b} are each independently selected from hydrogen

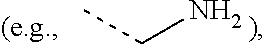

C1-6 alkyl, C2-6 alkenyl, C2-6 alkynyl, C3-7 carbocycle, C6-10 aryl, 5-10 membered heteroaryl, and 5-10 membered heterocyclyl, as defined herein.

The term “attached,” as used herein, refers to the state of two things being joined, fastened, adhered, connected or bound to each other, either directly or indirectly. As an example, an oligonucleotide primer can be chemically attached to a polymeric hydrogel via a covalent or non-covalent bond (see, e.g., the primers 42A, 42B attached to the polymeric hydrogel 32 in FIG. 1B, FIG. 1C, and in FIG. 2A through FIG. 2D). As another example, a first substrate may be physically attached to either a lid or to a second substrate via the interposer that is in contact with a bonding material 26A, 26B, 26C, 28 (see, e.g., the first substrate 14 or 16 attached to the lid 22 or the second substrate 24 via the interposer 30 in FIG. 1B and in FIG. 1C).

An “azide” or “azido” functional group refers to —N₃.

The term “bonding material,” as used herein, refers to a material that is capable of attaching a (first) substrate to either a lid or to a second substrate via an intervening interposer. In some instances, the term refers to a light-switchable bonding material, as will be defined herein. In other instances, the term refers to a solvent-responsive bonding material, as will be defined herein. As will be described, the term “switchable” refers to these bonding materials' ability to be chemically modified upon exposure to a preselected reaction condition, such as exposure to a preselected light wavelength, exposure to a preselected solvent for a predetermined time, or exposure to a preselected temperature for a predetermined time. Alternatively, the term “bonding material” may refer to a non-switchable bonding material, meaning that the bonding material does not undergo a chemical change/modification upon exposure to a preselected reaction condition. In other words, the term “non-switchable bonding material” refers to a material that is selected to be inert to a reaction condition that will be used to modify a separate, switchable bonding material.

The term “bonding region,” as used herein, refers to an area of a material (e.g., a substrate or a lid) that is (or that is to be) attached to another material. The term may further refer to individual components used to bond the materials together, such as interposers, bonding materials (e.g., switchable and non-switchable bonding materials, as defined herein), and the like. In the example flow cells disclosed herein that include two substrates attached to one another, each of the two substrates may include a respective bonding region (see, e.g., FIG. 2C and FIG. 2D, each of which depicts a first substrate 14, 16 having first bonding region(s) 40 and a second substrate 24 having second bonding region(s) 40′). In the example flow cells disclosed herein that include a first substrate attached to a lid, both the first substrate and the lid may include a respective bonding region (see, e.g., FIG. 2A and FIG. 2B, each of which depicts a first substrate 14, 16 having first bonding region(s) 40 and a lid 22 having second bonding region(s) 40′). The bonding region(s) may further include nanodepressions defined therein, such that a bonding material that is in contact with the interposer is also disposed within the nanodepressions (see, e.g., the bonding regions 40, 40′ including the depressions 36′ in FIG. 2C, where the bonding material 26A, 26B, 26C, or 28 at least partially fills the depressions 36′).

The term “carbocycle,” as used herein, refers to a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

The terms “carboxylic acid” or “carboxyl,” as used herein, refer to

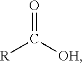

where R is an alkyl group, an alkenyl group, an aryl group, or another substituent.

The term “cycloalkyl,” as used herein, refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

The terms “cycloalkenyl” or “cycloalkane,” as used herein, refer to a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

The terms “cycloalkynyl” or “cycloalkyne,” as used herein, refer to a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, slot die coating, puddle dispensing, flow through coating, flow through deposition, aerosol printing, screen printing, microcontact printing, inkjet printing, or the like.

The term “depression,” as used herein, refers to a discrete concave feature defined in a substrate and having a surface opening. In some instances, the term refers to depressions that are defined within an active region of a substrate and that include surface chemistry (i.e., a polymeric hydrogel and primers attached thereto) therein (see the depressions 36 in the active region 38 in FIG. 2A through FIG. 2D). In other instances, the term refers to depressions that are defined within a bonding region of a flow cell and that include a bonding material therein (see the depressions 36′ in the bonding region(s) 40 in FIG. 2B through FIG. 2D, which do not include surface chemistry therein but do include the bonding material 26A, 26B, 26C, or 28). Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As examples, the depression can be a well or two interconnected wells.

The term “each,” when used herein in reference to a collection of items, is intended to identify an individual item in the collection, but may not necessarily refer to every item in the collection. Exceptions can occur if explicit disclosure or context clearly dictates otherwise.

The term “epoxy,” as used herein, refers to

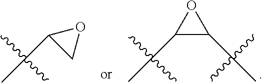

The term “flow cell,” as used herein, is intended to refer to a vessel having an enclosed flow channel and active region where a reaction can be carried out. A flow cell with an enclosed channel also includes an inlet for delivering reagent(s) to the channel and an outlet for removing reagent(s) from the channel. In some examples, the flow cell enables the detection of the reaction that occurs therein. For example, the flow cell can include one or more transparent surfaces allowing for the optical detection of arrays, optically labeled molecules, or the like.

The terms “flow channel” or “channel,” as used herein, refer to an area defined between two bonded components, such as lids and/or substrates. The flow channel (channel) can selectively receive a liquid sample, reagents, etc. In some examples disclosed herein, the flow channel is defined between two substrates, and the flow channel is in fluid communication with surface chemistry disposed within (an) active region(s) defined between the two substrates. In other examples disclosed herein, the flow channel is defined between one substrate and a lid, and the flow channel is in fluid communication with surface chemistry within (an) active region(s) defined between the substrate and the lid.

The term “heteroaryl,” as used herein, refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

The term “heterocycle,” as used herein, refers to a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The terms “hydrazine” or “hydrazinyl,” as used herein, refer to a —NHNH₂ group.

The terms “hydrazone” or “hydrazonyl,” as used herein, refer to a

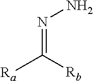

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

The terms “hydroxy” or “hydroxyl,” as used herein, refer to an —OH group.

The terms “hydrogel” or “polymeric hydrogel,” as used herein, refer to a semi-rigid polymer that is permeable to liquids and/or gases. The hydrogel can swell when liquid (e.g., water) is taken up and that can contract when liquid is removed, e.g., by drying. While a hydrogel may absorb water, it is not water-soluble. In some instances, the hydrogel is positioned within an active region of a flow cell and has primers attached thereto. In some examples, the polymeric hydrogel is referred to as being a “pre-grafted polymeric hydrogel,” meaning that primers (of a primer set) are grafted/attached to the polymeric hydrogel before the polymeric hydrogel is deposited over a substrate surface. In other examples, the polymeric hydrogel is referred to as being a “non-pre-grafted polymeric hydrogel,” meaning that primers (of a primer set) are grafted/attached to the polymeric hydrogel after the polymeric hydrogel is deposited over the substrate surface. In further examples, the hydrogel is used as a solvent-responsive bonding material, is positioned at the bonding region of a flow cell, and does not have primers attached thereto. These examples of the hydrogel may be referred to herein as a “hydrogel adhesive.”

The term “interposer,” as used herein, refers to a material that is bonded between two flow cell components (e.g., substrates and/or lids) together. The interposer may act as a spacer layer. The interposer includes any suitable material that can be coated with a switchable or non-switchable bonding material.

The term “interstitial region,” as used herein, refers to an area of a substrate that separates individual depressions or functionalized pads from one another. In some instances, the interstitial regions separate depressions or functionalized pads within an individual active region of a substrate from other depressions or functionalized pads within the active region. In other instances, the interstitial regions separate individual depressions within an individual bonding region of a substrate from other depressions within the bonding region. The separation provided by (an) interstitial region(s) can be partial or full separation.

The term “light-switchable bonding material,” as used herein, refers to a material that undergoes a chemical change upon exposure to a preselected light wavelength, where the chemical change alters the material's physical properties. As an example, the light-switchable bonding material may undergo an increase or a decrease in adhesive properties upon exposure to the preselected light wavelength.

The term “nitrile oxide,” as used herein, refers to a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

The term “nitrone,” as used herein, refers to a

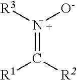

group in which R¹, R², and R³ may be any of the Rₐ and R_{b} groups defined herein, except that R³ is not hydrogen (H).

The term “nucleotide,” as used herein, refers to a nitrogen-containing heterocyclic base, a sugar, and one or more phosphate groups. Nucleotides are monomeric units of a nucleic acid sequence. In RNA (ribonucleic acid), the sugar is a ribose, and in DNA (deoxyribonucleic acid), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

The term “over” may refer to a component or material that is positioned directly on another component or material. When one component or material is “directly on” another, the two are in direct physical contact with each other. For example, in FIG. 1B, the polymeric hydrogel 32 is “over” the layer 20 (when a multi-layer substrate 16 is utilized) or “over” the single layer substrate 14, such that the polymeric hydrogel 32 is in direct physical contact with the layer 20 or the substrate 14.

The term “over” may alternatively refer to one component or material that is positioned indirectly on another component or material. By “indirectly on,” it is meant that a gap or an additional component or material is positioned between two components or materials. For example, in FIG. 1B, the interposer 30 is positioned “over,” but indirectly on, the substrate 14, 16. More specifically, the interposer 30 is indirectly on the substrate 14, 16 because the bonding material 26A, 26B, 26C, or 28 is positioned between the substrate 14, 16 and the interposer 30.

The term “patterned structure,” as used herein, refers to a substrate that includes (an) active region(s) and (a) bonding region(s), where the active region(s) include(s) a defined (e.g., patterned) architecture. In some examples, the term refers to a substrate that is patterned with depressions (as shown in FIG. 2A through FIG. 2D). In other examples, the term refers to a substrate that is patterned with functionalized pads (see the functionalized pads 27 shown in FIG. 1C). The patterned structure may be exposed to patterning techniques (e.g., etching, lithography, etc.) and/or coating techniques in order to generate the pattern(s) within the active region(s). In contrast, the term “unpatterned structure” refers to a substrate that includes an active region, but that does not include depressions or functionalized pads in the active region (see, e.g., the unpatterned structure in FIG. 1B, which depicts a lane 34 in a flow cell).

The term “polyhedral oligomeric silsesquioxane” (an example of which is commercially available under the tradename “POSS”), as used herein, refers to a chemical composition that is a hybrid intermediate (e.g., RSiO_{1.5}) between that of silica (SiO₂) and silicone (R₂SiO). An example of polyhedral oligomeric silsesquioxane may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for POSS include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

The term “primer,” as used herein, refers to a single stranded nucleic acid sequence (e.g., single stranded DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of a polymeric hydrogel. The primer length can be any number of bases long and can include a variety of non-natural nucleotides. In an example, the sequencing primer is a short strand, ranging from 10 to 60 bases, or from 20 to 40 bases.

The term “solvent-responsive bonding material,” as used herein, refers to a material that undergoes a chemical change during a timed exposure to a preselected solvent, where the chemical change alters the physical properties of the material (and thus the solvent-responsive bonding may be referred to herein as a “switchable” bonding material). As an example, the solvent-responsive bonding material may undergo a decrease in adhesive properties upon exposure to the preselected solvent for a predetermined time period. In some examples disclosed herein, the solvent-responsive material is a hydrogel-based material (e.g., a hydrogel adhesive). In other examples disclosed herein, the solvent-responsive material is a non-hydrogel-based material.

The term “substrate” may be used herein in conjunction with the term “single layer substrate” or “multi-layer substrate.” A single layer substrate refers to a one layered support material (see, e.g., the substrate 14 depicted in FIG. 1B and FIG. 1C and in FIG. 2A through FIG. 2D). A multi-layered substrate includes at least two layers. For example, the multi-layer substrate may include a base support with an additional layer thereon (see, e.g., the substrate 16 including the base support 18 and the layer 20 over the base support 18 depicted in FIG. 1B and FIGS. 1C and 1n FIG. 2A through FIG. 2D).

The term “surface chemistry,” as defined herein, refers to a polymeric hydrogel (as defined herein) and at least one primer attached thereto. Surface chemistry may be positioned within one or more active regions of a flow cell. In some instances, the surface chemistry is positioned within a lane defined in a substrate (see the active region 38 including the lane 34 in FIG. 1B). In other instances, the surface chemistry is provided in the form of functionalized pads on a substrate (see the functionalized pads 27 that make up the active region 38 in FIG. 1C). In still other instances, the surface chemistry is positioned within depressions of an active region (see the active region(s) 38 in FIG. 2A through FIG. 2D, each active region 38 including depressions 36).

The term “tantalum pentoxide,” as used herein, refers to the inorganic compound with the formula Ta₂O₅. This compound is transparent, having a transmittance ranging from about 0.25 (25%) to 1 (100%), to wavelengths ranging from about 0.35 μm (350 nm) to at least 1.8 μm (1800 nm). A “tantalum pentoxide substrate” may comprise, consist essentially of, or consist of Ta₂O₅. In examples where it is desirable for the tantalum pentoxide substrate to transmit electromagnetic energy having any of these wavelengths, the substrate may consist of Ta₂O₅ or may comprise or consist essentially of Ta₂O₅ and other components that will not interfere with the desired transmittance of the substrate.

A “thiol” functional group refers to —SH.

The terms “tetrazine” and “tetrazinyl,” as used herein, refer to six-membered heteroaryl group comprising four nitrogen atoms. Tetrazine can be optionally substituted.

The term “tetrazole,” as used herein, refers to five-membered heterocyclic group including four nitrogen atoms. Tetrazole can be optionally substituted.

The term “transparent,” as used herein when describing a material (e.g., substrate, layer, etc.), means that that the material allows light of a particular wavelength or range of wavelengths to pass through. Transparency may be quantified using transmittance, i.e., the ratio of light energy falling on a body to that transmitted through the body. The transmittance of a transparent material will depend upon the thickness of the material and the wavelength of light. In the examples disclosed herein, the transmittance of the transparent material may range from 0.25 (25%) to 1 (100%). The material may be a pure material, a material with some impurities, or a mixture of materials, as long as the resulting material is capable of the desired transmittance. Additionally, depending upon the transmittance of the material, the time for light exposure and/or the output power of the light source may be increased or decreased to deliver a suitable dose of light energy through the transparent material to achieve the desired effect. For example, a material may be transparent to a light source that is used to modify a light-switchable bonding material. As another example, a material may be transparent to an excitation light source that is used during a sequencing operation.

### Flow Cells and Flow Cell Formation

Some examples of the flow cell disclosed herein generally include a first substrate including an active region and a bonding region that is spatially separate from the active region; a polymeric hydrogel positioned at the active region; an interposer having a first side that is attached to the bonding region and having a second side that is opposed to the first side; a light-switchable bonding material in contact with at least one of the first side or the second side; and a lid or a second substrate attached to the second side of the interposer.

Other examples of the flow cell disclosed herein generally include a first substrate including an active region and a bonding region that is spatially separate from the active region; a polymeric hydrogel positioned at the active region; an interposer having a first side that is attached to the bonding region of the first substrate and having a second side that is opposed to the first side; a solvent-responsive bonding material on at least one of the first side or the second side; and a lid or a second substrate attached to the second side of the interposer.

FIG. 1A depicts an example of the flow cell 10 from a top view. The flow cell 10 shown in FIG. 1A includes (a) patterned structure(s), (an) unpatterned structure(s), and/or a lid. An example of an unpatterned structure, including a lane 34 defined therein, bonded to a lid 22 or to a second substrate 24 is shown in FIG. 1B. In FIG. 1B, the lane 34 defines an active region 38 of the flow cell 10. Another example of a patterned structure, including a plurality of functionalized pads 27, bonded to a lid 22 or to a second substrate 24 is shown in FIG. 1C. In FIG. 1C, the functionalized pads 27 define an active region 38 of the flow cell 10. Still another example of a patterned structure, including a plurality of depressions 36 defined therein, bonded to a lid 22 or to a second substrate 24 will later be described in reference to FIG. 2A through FIG. 2D.

The unpatterned structure and/or the patterned structure that is used in the flow cell 10 includes a single layer substrate 14 or a multi-layer substrate 16. The multi-layer substrate 16, when included in the flow cell 10, includes a base support 18 and an additional layer 20 positioned over (and in direct physical contact with) the base support 18.

Examples of suitable materials for the single layer substrate 14 include siloxanes (e.g., epoxy siloxane), glass, modified or functionalized glass, polymeric materials (including acrylics, polystyrene and copolymers of styrene and other materials, polypropylene, polyethylene, polybutylene, polyurethanes, polytetrafluoroethylene (such as TEFLON® from Chemours), polyethylene terephthalate (PET), polycarbonate, cyclic olefins/cyclo-olefin polymers (COP) (such as ZEONOR® from Zeon), polyimides, nylon (polyamides), etc.), ceramics/ceramic oxides, silica (i.e., silicon dioxide (SiO₂)), fused silica, or silica-based materials, aluminum silicate, silicon and modified silicon (e.g., boron doped p+ silicon), silicon nitride (Si₃N₄), tantalum pentoxide (Ta₂O₅) or other tantalum oxide(s) (TaOₓ), hafnium oxide (HfO₂), carbon, metals, resins, or the like. Examples of suitable resins include inorganic oxides, such as tantalum pentoxide (e.g., Ta₂O₅) or other tantalum oxide(s) (TaOₓ), aluminum oxide (e.g., Al₂O₃), silicon oxide (e.g., SiO₂), hafnium oxide (e.g., HfO₂), indium tin oxide, titanium dioxide, etc., or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. It is to be understood that the material of the substrate 14 may be any material that can be etched, imprinted, or manipulated to form the lane 34 shown in FIG. 1B (or to form the depressions 36 that will be described in reference to FIG. 2A through FIG. 2D). It is to be further understood that the material of the substrate 14 may be any material that can be patterned with the functionalized pads 27 shown in FIG. 1C.

As mentioned, examples of the multi-layer substrate 16 include the base support 18 and at least one other layer 20 positioned thereon. Any example of the material of the single layer substrate 14 provided herein may be used as the material for the base support 18 of the multi-layer substrate 16. Examples of suitable materials for the layer 20 include inorganic oxides, such as tantalum oxide (e.g., Ta₂O₅), aluminum oxide (e.g., Al₂O₃), silicon oxide (e.g., SiO₂), or hafnium oxide (e.g., HfO₂), or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. It is to be understood that in examples of the flow cell 10 that include the substrate 16, the layer 20 (positioned on the base support 18) may be any material that can be etched, imprinted, or manipulated to form the lane 34 shown in FIG. 1B (or to form the depressions 36 that will be described in reference to FIG. 2A through FIG. 2D). It is to be further understood that the material of the layer 20 may be any material that can be patterned with the functionalized pads 27 shown in FIG. 1C.

Suitable deposition techniques for the material(s) of the layer 20 (when the multi-layer substrate 16 is used) include chemical vapor deposition, dip coating, dunk coating, spin coating, spray coating, puddle dispensing, ultrasonic spray coating, doctor blade coating, aerosol printing, screen printing, microcontact printing, etc. It is to be understood that the deposition technique(s) that is/are used for the layer 20 may depend, in part, upon the material used to form the layer 20.

The single layer substrate 14 or the base support 18 (of the multi-layer substrate 16) may be a circular sheet, a panel, a wafer, a die etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). For example, a die may have a width ranging from about 0.1 mm to about 10 mm. While example dimensions have been provided, it is to be understood that a substrate 14 or base support 18 with any suitable dimensions may be used.

The thickness of the layer 20 (when the substrate 16 is used) is variable. In examples of the flow cell 10 that include depressions 36 in an active region 38, the thickness of the layer 20 is greater than the desired depth of the depressions 36 formed in the active region 38. In examples of the flow cell 10 that include the lane 34 (as in FIG. 1B), the thickness of the layer 20 is greater than the desired depth for the lane 34 formed therein.

The x and y dimensions (e.g., diameter, length, width, etc.) of the layer 20 may be greater than, less than, or equal to the x and y dimensions, respectively, of the base support 18.

Suitable patterning techniques for the substrate 14 (or for the layer 20 of the substrate 16) include photolithography, nanoimprint lithography (NIL), stamping techniques, embossing techniques, molding techniques, microetching techniques, etc. It is to be understood that the patterning technique(s) that is/are used may depend, in part, upon the material used to form the substrate 14 or to form the layer 20 of the substrate 16.

As shown in FIG. 1B and FIG. 1C, the substrate 14, 16 that is used in the flow cell 10 is bonded to the lid 22 or to the second substrate 24 via the interposer 30 at bonding regions 40, 40′. In particular, the interposer 30 has a first side S₁ that is attached to the first bonding region 40 of the substrate 14, 16, and the first side S₁ of the interposer 30 is in contact with a light-switchable bonding material 26A, a solvent-responsive bonding material 26B, a heat-responsive bonding material 26C, or a non-switchable bonding material 28. Further, the interposer 30 has a second side S₂ that is opposed to the first side S₁ and that is attached to the lid 22 or to the second substrate 24 (e.g., at a second bonding region 40′). The second side S₂ of the interposer 30 is also in contact with the light-switchable bonding material 26A, the solvent-responsive bonding material 26B, the heat-responsive bonding material 26C, or the non-switchable bonding material 28.

The light-switchable bonding material 26A, when included in the flow cell 10, may be any material that undergoes a chemical reaction resulting in increased or decreased properties of adhesion (within the light-switchable bonding material 26A) upon exposure to a preselected wavelength of light. In some examples, the light-switchable bonding material 26A undergoes an increase in properties of adhesion upon exposure to a preselected light wavelength in the ultraviolet (UV) range and undergoes a decrease in properties of adhesion upon exposure to a preselected light wavelength in the visible light range. In other examples, the light-switchable bonding material 26A undergoes an increase in properties of adhesion upon exposure to a preselected light wavelength in the visible light range and undergoes a decrease in properties of adhesion upon exposure to a preselected light wavelength in the ultraviolet (UV) range. The light-switchable bonding material 26A may be selected from the group consisting of an epoxy acrylate, an acrylic polymer, silicone, a cyanoacrylate, a base polymer combined with an oligomer and a photoinitiator, and a combination thereof. Specific examples of the light-switchable bonding material 26A include poly(6-(4-(p-tolyldiazenyl)phenoxy)hexyl acrylate), a castor oil-based urethane oligomer that is sealed with hydroxylethyl acrylate, and an acrylic polymer formed from an aliphatic monomer containing an azobenzene moiety and a photoinitiator. In some instances, the light-switchable bonding material 26A includes a photo-cleavable group, such as an arylcarbonylmethyl group, a nitroaryl group, coumarin, an arylmethyl group, and others. A specific example of a photo-cleavable group is poly(6-(4-(p-tolydiazenyl)phenoxy)hexyl acrylate). In some examples, the light-switchable bonding material 26A includes multifunctional crosslinkers, or materials having photo-reactive side chains (e.g., pendant vinyl groups). In further examples, the light-switchable bonding material 26A is a light to heat converting elastomer that undergoes a decrease in adhesive properties upon exposure to light, where energy from the light is converted to heat upon exposure to preselected wavelengths. In an example, the light to heat converting elastomer is a 3M™ OneFilm product. As shown in FIG. 1B and FIG. 1C and as described herein, the light-switchable bonding material 26A may be positioned between the substrate 14, 16 and the interposer 30 (e.g., at the first bonding region 40), and/or between the interposer 30 and the lid 22 or second substrate 24 (e.g., at the second bonding region 40′).

The solvent-responsive bonding material 26B, when included in the flow cell 10, may be any material that is capable of undergoing a chemical reaction resulting in decreased properties of adhesion (within the solvent-responsive bonding material 26B) during a timed exposure to a preselected solvent. In some examples, the solvent-responsive bonding material 26B is a hydrogel adhesive. In other examples, the solvent-responsive bonding material 26B is non-hydrogel-based (e.g., an acrylic adhesive). The solvent-responsive bonding material 26B may be selected from the group consisting of a poly(2-hydroxyethyl methacrylate) (PHEMA), an ethylene glycol dimethacrylate (EGDMA) cross-linked PHEMA hydrogel, an acrylate-based polymer, and a combination thereof. It is to be understood that some of the light-switchable bonding materials 26A described herein may undergo a physical or chemical change during a timed exposure to a suitable solvent as well.

As one example, the solvent-responsive bonding material 26B may experience a decrease in adhesive properties upon exposure to a suitable solvent, such as isopropyl alcohol.

As shown in FIG. 1B and FIG. 1C and as described herein, the solvent-responsive bonding material 26B may be positioned between the substrate 14, 16 and the interposer 30 (e.g., at the first bonding region 40), and/or between the interposer 30 and the lid 22 or second substrate 24 (e.g., at the second bonding region 40′).

The heat-responsive bonding material 26C, when included in the flow cell 10, may be any material that is capable of undergoing a chemical reaction resulting in decreased properties of adhesion (within the heat-responsive bonding material 26C) during a timed exposure to a predetermined temperature or range of temperatures.

In some examples, the heat-responsive bonding material 26C has microcapsules dispersed (or dissolved) therein, where the microcapsules include a thermoplastic shell and a liquid interior. In these examples, when the heat-responsive bonding material 26C is exposed to the predetermined temperature, the thermoplastic shell of the microspheres softens and gasification of the liquid occurs. The gasification of the liquid increases the volume of the heat-responsive bonding material 26C, thereby reducing the adhesive strength of the heat-responsive bonding material and facilitating de-bonding. The thermoplastic shell may include poly(acrylonitrile), poly(vinylidene chloride), polyolefins, poly(methyl methacrylate), or another suitable thermoplastic material. The liquid encapsulated by the thermoplastic shell may include a hydrocarbon, such as i-butane, n-hexane, petroleum ether, or another suitable hydrocarbon that will undergo gasification at a predetermined temperature.

The heat-responsive bonding material 26C may be selected from the group consisting of a urethane including substituted urea linkages, a urethane having substituted peroxide linkages, a block copolymer with degradable polyperoxide segments, a thermoplastic polymer shell encapsulating a hydrocarbon, and a blocked polyisocyanate crosslinker. In an example, the heat-responsive bonding material 26C is selected from the group consisting of 3M™ thermally conductive adhesive tape 8805, NITTO™ heat-releasable tape, and HENCKEL™ Loctite thermal release tape. As shown in FIG. 1B and FIG. 1C and as described herein, the heat-responsive bonding material 26C may be positioned between the substrate 14, 16 and the interposer 30 (e.g., at the first bonding region 40), and/or between the interposer 30 and the lid 22 or second substrate 24 (e.g., at the second bonding region 40′).

The non-switchable bonding material 28, when included in the flow cell 10, may be any material that is selected to be inert to a preselected reaction condition, where the preselected reaction condition will be used to modify the light-switchable bonding material 26A, the solvent-responsive bonding material 26B, or the heat responsive bonding material 26C used in the flow cell 10. In some examples, such as when the light-switchable bonding material 26A is included in the flow cell 10, the non-switchable bonding material 28 is selected to be inert to the preselected light wavelength that will be used to modify the light-switchable bonding material 26A. In other examples, such as when the solvent-responsive bonding material 26B is included in the flow cell 10, the non-switchable bonding material 28 is selected to be inert to the preselected solvent or kinetically disfavored for reaction with the preselected solvent that will be used to modify the solvent-responsive bonding material 26B. In further examples, such as when the heat-responsive bonding material 26C is included in the flow cell 10, the non-switchable bonding material 28 is selected to be inert to the predetermined temperature that will be used to modify the heat-responsive coating 26C. The non-switchable bonding material 28 may include the materials described herein in reference to the light-switchable bonding material 26A, the solvent-responsive bonding material 26B, or the heat-responsive bonding material 26C, depending on the reaction conditions that will be used to modify the other bonding material(s) 26A, 26B, or 26C included in the flow cell 10. As shown in FIG. 1B and FIG. 1C, the non-switchable bonding material 28 may be positioned between the substrate 14, 16 and the interposer 30 (e.g., at the first bonding region 40), or between the interposer 30 and the lid 22 or second substrate 24 (e.g., at the second bonding region 40′).

The light-switchable bonding material 26A, the solvent-responsive bonding material 26B, the heat-responsive bonding material 26C, and the non-switchable bonding material 28 are applied as a layer having a thickness. The thickness of each of the layer(s) of the bonding material(s) 26A, 26B, 26C, 28 is variable. In examples, the thickness of the layer(s) of the bonding material(s) 26A, 26B, 26C, 28 ranges from about 0.1 μm to about 50 μm. In a specific example, the thickness of the layer(s) of the bonding material(s) 26A, 26B, 26C, 28 is about 12.5 μm.

The interposer 30 that is used to attach the substrate 14, 16 to the lid 22 or to the second substrate 24 may include any material that will seal portions of the substrate 14, 16 to the lid 22 or to the second substrate 24. As examples, the interposer 30 may be a polymer (e.g., PET with carbon black), glass, metal, or any other suitable material that can be used to attach the substrate 14, 16 to the lid 22 or to the second substrate 24 using the bonding material 26A, 26B, 26C, or 28. The thickness of the interposer 30 may range from about 10 μm to about 150 μm. In a specific example, the thickness of the interposer 30 ranges from about 50 μm to about 75 μm.

The lid 22, when included in the flow cell, 10 may be any suitable material that can be used to seal an interior of the flow cell 10 and create an enclosed space (e.g., to create a flow channel 12 in communication with an active region 38). In examples, the lid 22 includes a material that is transparent to an excitation light source, or that is transparent to a light source suitable for chemically modifying the light-switchable bonding material 26A. As specific examples, the lid 22 may be a glass lid or a polymer lid. A commercially available example of a suitable borosilicate glass is D 263®, available from Schott North America Inc. Commercially available examples of suitable polymer materials, namely cyclo olefin polymers, are the ZEONOR® products available from Zeon Chemicals L.P.

The second substrate 24, when included in the flow cell 10, may include any unpatterned or patterned structure described herein and may be used to create an enclosed space (e.g., to create a flow channel 12 in communication with an active region 38). For example, the second substrate 24 may be an unpatterned structure including a lane 34 that forms a (second) active region 38′ (not shown in FIG. 1A through FIG. 1C), similar to the substrate 14, 16 including the lane 34 defined therein that forms the active region 38 in FIG. 1B. As another example, the second substrate 24 may be a patterned structure including a plurality of functionalized pads 27 that form a (second) active region 38′ (not shown in FIG. 1A through FIG. 1C), similar to the substrate 14, 16 having the functionalized pads 27 thereon that form the active region 38 in FIG. 1C. As still another example, the second substrate 24 may be a patterned structure including a plurality of depressions 36 defined therein that form a (second) active region 38′, which will be described further in reference to FIG. 2C and FIG. 2D.

It is to be understood that any combination of the light-switchable bonding material 26A, the solvent-responsive bonding material 26B, the heat-responsive bonding material 26C, and the non-switchable bonding material 28 may be included in the flow cell 10. Various configurations of the bonding materials 26A, 26B, 26C, 28 in the flow cell 10 will now be described.

In some examples, the light-switchable bonding material 26A is in contact with the first side S₁ or the second side S₂ of the interposer 30, and the non-switchable bonding material 28 is in contact with the other of the second side S₂ or the first side S₁ of the interposer 30.

In one of these examples, the light-switchable bonding material 26A is in contact with the first side S₁ of the interposer 30 and attaches the first side S₁ to the bonding region 40. In this same example, the non-switchable bonding material 28 is in contact with the second side S₂ of the interposer 30, and the non-switchable bonding material 28 attaches the second side S₂ to the lid 22 or to the second substrate 24 (e.g., at the second bonding region 40′).

In another of these examples, the light-switchable bonding material 26A is in contact with the second side S₂ of the interposer 30 and attaches the second side S₂ to the lid 22 or to the second substrate 24 (e.g., at the second bonding region 40′). In this same example, the non-switchable bonding material 28 is in contact with the first side S₁ of the interposer 30 and the non-switchable bonding material 28 attaches the first side S₁ to the (first) bonding region 40.

In some other examples, the solvent-responsive bonding material 26B is in contact with the first side S₁ or the second side S₂ of the interposer 30, and the non-switchable bonding material 28 is in contact with the other of the second side S₂ or the first side S₁ of the interposer 30.

In one of these examples, the solvent-responsive bonding material 26B is in contact with the first side S₁ of the interposer 30 and attaches the first side S₁ to the bonding region 40. In this same example, the non-switchable bonding material 28 is in contact with the second side S₂ of the interposer 30 and attaches the second side S₂ to the lid 22 or to the second substrate 24 (e.g., at the second bonding region 40′).

In another of these examples, the solvent-responsive bonding material 26B is in contact with the second side S₂ of the interposer 30 and attaches the second side S₂ to the lid 22 or to the second substrate 24 (e.g., at the second bonding region 40′). In this same example, the non-switchable bonding material 28 is in contact with the first side S₁ of the interposer 30, and the non-switchable bonding material 28 attaches the first side S₁ to the bonding region 40.

In further examples, the heat-responsive bonding material 26C is in contact with the first side S₁ or the second side S₂ of the interposer 30, and the non-switchable bonding material 28 is in contact with the other of the second side S₂ or the first side S₁ of the interposer 30.

In one of these examples, the heat-responsive bonding material 26C is in contact with the first side S₁ of the interposer 30 and attaches the first side S₁ to the bonding region 40. In this same example, the non-switchable bonding material 28 is in contact with the second side S₂ of the interposer 30 and attaches the second side S₂ to the lid 22 or to the second substrate 24 (e.g., at the second bonding region 40′).

In another of these examples, the heat-responsive bonding material 26C is in contact with the second side S₂ of the interposer 30 and attaches the second side S₂ to the lid 22 or to the second substrate 24 (e.g., at the second bonding region 40′). In this same example, the non-switchable bonding material 28 is in contact with the first side S₁ of the interposer 30, and the non-switchable bonding material 28 attaches the first side S₁ to the bonding region 40.

In still further examples, the light-switchable bonding material 26A, the solvent-responsive bonding material 26B, or the heat-responsive bonding material 26C is in contact with both the first side S₁ and the second side S₂ of the interposer 30.

As such, in one of these examples, the light-switchable bonding material 26A: (i) is in contact with both the first side S₁ and the second side S₂ of the interposer 30, (ii) attaches the first side S₁ to the bonding region 40, and (iii) attaches the second side S₂ to the lid 22 or to the second substrate 24 (e.g., at the second bonding region 40′).

In another of these examples, the solvent-responsive bonding material 26B: (i) is in contact with both the first side S₁ and the second side S₂ of the interposer 30, (ii) attaches the first side S₁ to the bonding region 40, and (iii) attaches the second side S₂ to the lid 22 or to the second substrate 24 (e.g., at the second bonding region 40′).

In yet another of these examples, the heat-responsive bonding material 26C: (i) is in contact with both the first side S₁ and the second side S₂ of the interposer 30, (ii) attaches the first side S₁ to the bonding region 40, and (iii) attaches the second side S₂ to the lid 22 or to the second substrate 24 (e.g., at the second bonding region 40′).

Regardless of the configuration of the bonding materials 26A, 26B, 26C, or 28 that are included in the flow cell 10, as further shown in FIG. 1A, the flow cell 10 may include one or more flow channel(s) 12. Each flow channel 12 is an enclosed space that is defined by the substrate 14, 16, the interposer 30, the bonding material(s) 26A, 26B, 26C, and/or 28, and the lid 22 or the second substrate 24, and each flow channel 12 is in fluid communication with at least one active region 38 of the flow cell 10.

The example flow cell 10 shown in FIG. 1A includes eight flow channels 12. While eight flow channels 12 are shown in FIG. 1A, it is to be understood that any number of flow channels 12 may be included in the flow cell 10 (e.g., a single flow channel 12, four flow channels 12, twelve flow channels 12, etc.). When multiple flow channels 12 are included in the flow cell 10, each flow channel 12 may be isolated from another flow channel 12 so that fluid introduced into one flow channel 12 does not flow into (an) adjacent flow channel(s) 12.

Regardless of the number of flow channels 12 that are included in the flow cell 10, each flow channel 12 may have any desirable shape. In an example, the flow channel 12 has a substantially rectangular configuration with curved ends (as shown in FIG. 1A). The length of the flow channel 12 depends, in part, upon the size of the substrate 14 or 16 used to form the unpatterned or patterned structure(s) included in the flow cell 10. The width of each flow channel 12 depends, in part, upon the size of the substrate 14 or 16 used to form the unpatterned or patterned structure(s) included in the flow cell, the desired number of flow channels 12, and the desired space at a perimeter of the unpatterned or patterned structure(s) included in the flow cell 10.

The depth of each flow channel 12 may range from about 10 μm to about 400 μm. In an example, the depth ranges from about 10 μm to about 30 μm. In another example, the depth is about 5 μm or less. It is to be understood that the depth of the flow channel(s) 12 may be greater than, less than, or between the values specified above.

Each flow channel 12 that is included in the flow cell 10 may be in fluid communication with an inlet and an outlet (not shown in FIG. 1A through FIG. 1C). The inlet and outlet of each flow channel 12 may be positioned at opposed ends of the flow cell 10. The inlets and outlets of the respective flow channels 12 may alternatively be positioned anywhere along the length and width of the flow channel 12 that enables desirable fluid flow.

The inlet allows fluid(s) to be introduced into the flow channel 12, and the outlet allows fluid(s) to be extracted from the flow channel 12. Each of the inlet(s) and outlet(s) is/are fluidly connected to a fluidic control system (e.g., reservoirs, pumps, valves, waste containers, and the like) that controls fluid introduction and expulsion. Some examples of the fluids that may be introduced into the flow channel(s) 12 include reaction components (e.g., DNA library templates, polymerases, sequencing primers, nucleotides, etc.), washing solutions, etc.

As shown in FIG. 1B and FIG. 1C and as described, each flow channel 12 is in fluid communication with an active region 38. Each active region 38 includes surface chemistry (i.e., a polymeric hydrogel 32 and one or more primers 42A, 42B attached thereto). In FIG. 1B, the lane 34 includes the polymeric hydrogel 32 having primers 42A, 42B attached thereto. In FIG. 1C, the functionalized pads 27 are composed of the polymeric hydrogel 32 and have the primers 42A, 42B attached thereto (and thus the functionalized pads 27 make up the surface chemistry). As will be described in reference to FIG. 2A through FIG. 2D, when the flow cell 10 includes a plurality of depressions 36 within an active region 38, the polymeric hydrogel 32 and the primers 42A, 42B are positioned within each of the plurality of depressions 36 in the active region 38.

The polymeric hydrogel 32 may be any gel material that can swell when liquid is taken up and that can contract when liquid is removed, e.g., by drying. Further, the polymeric hydrogel 32 may be any material that is capable of attaching oligonucleotide primers thereto. In an example, the polymeric hydrogel 32 includes an acrylamide copolymer, such as poly N-(5-azidoacetamidylpentyl)acrylamide-co-acrylamide, PAZAM. PAZAM and some other forms of the acrylamide copolymer are represented by the following structure (I):

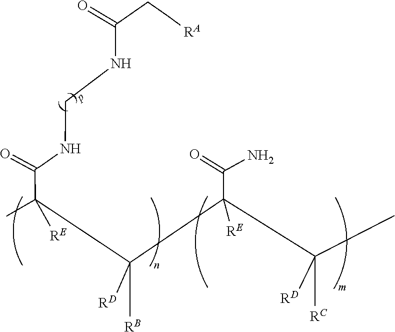

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and m is an integer in the range of 1 to 100,000.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa. In a specific example, the molecular weight of the acrylamide copolymer is about 312 kDa.

In some examples, PAZAM and other forms of the acrylamide copolymer are linear polymers. In some other examples, PAZAM and other forms of the acrylamide copolymer are lightly cross-linked polymers.

In other examples, the gel material may be a variation of the structure (I). In one example, the acrylamide unit may be replaced with N,N-dimethylacrylamide (

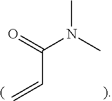

In this example, the acrylamide unit in structure (I) may be replaced with

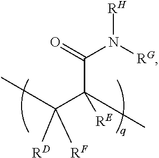

Where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

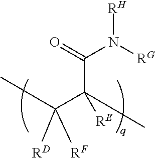

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel 32, the recurring “n” feature in structure (I) may be replaced with a monomer including a heterocyclic azido group having structure (II):

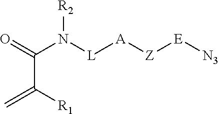

wherein R¹ is H or a C1-C6 alkyl; R₂ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the polymeric hydrogel 32 may include a recurring unit of each of structure (III) and (IV):

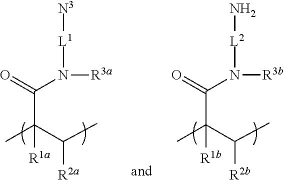

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each of L¹ and L² is independently selected from an optionally substituted alkylene linker or an optionally substituted heteroalkylene linker.

It is to be understood that other polymeric hydrogel materials may be used for the polymeric hydrogel 32, with the understanding that the hydrogel materials are suitable for grafting primers 42A, 42B thereto. Some additional examples of suitable materials for the polymeric hydrogel 32 include functionalized polysilanes, such as azido silane, amine functionalized silane, or any other polysilane having functional groups that can attach the desired biological reactants/primers. Other examples of suitable polymeric hydrogels 32 include those having a polymer mesh structure, such as gelatin (which includes amine groups) or other polypeptides; or a cross-linked polymer structure, such as an azidolyzed version of silane free acrylamide (SFA).

The polymeric hydrogel 32 may be formed using any suitable copolymerization process and may be deposited using any of the methods disclosed herein. For at least some of the deposition techniques, the polymeric hydrogel 32 may be incorporated into a mixture or solution, e.g., with water or with ethanol and water, and then applied within the lane 34, within the depressions 36 (not shown in FIG. 1B or FIG. 1C), or to form the functionalized pads 27. In some instances, the polymeric hydrogel 32 is a cured hydrogel (e.g., that has been cured using heat, UV/high energy light, or the like). In these instances, the polymeric hydrogel 32 may first be applied as the polymeric hydrogel mixture or solution (with the water and the ethanol) and then subsequently cured to form the polymeric hydrogel layer 32.

It is to be understood that some examples of the polymeric hydrogel 32 disclosed herein may also be used as the solvent-responsive bonding material 26B, when the solvent-responsive bonding material 26B is included.

The attachment of the polymeric hydrogel 32 to the substrate 14 or to the layer 20 of the multi-layer substrate 16 may be through covalent bonding. In some instances, the substrate 14 or the layer 20 (of the substrate 16) is activated to facilitate the covalent bonding of the polymeric hydrogel 32 to the substrate 14 or to the layer 20. Activation of the substrate 14 or layer 20, when performed, facilitates the attachment of the polymeric hydrogel 32 to the substrate 14 or layer 20 within the active region 38. Activation of the substrate 14 or the layer 20 may be accomplished via silanization or plasma ashing of the substrate 14 or layer 20 at the active region 38. Both silanization and plasma ashing will be described in more detail in regard to the methods disclosed herein.

As described, and as shown respectively in FIG. 1B and FIG. 1C, the polymeric hydrogel 32 within the lane 34 or forming the functionalized pads 27 may include primers 42A, 42B attached thereto. As such, some examples of the flow cell 10 include a plurality of primers 42A, 42B attached to the polymeric hydrogel 32. The primers 42A, 42B, when included as part of the flow cell 10, may form a primer set and may be different with respect to one another (e.g., in terms of the individual nucleotides that make up each of the primers 42A, 42B, or in terms of cleavage sites included in each of the primers 42A, 42B).

In an example, the primers 42A, 42B are utilized as nucleic acid probes that include respective target capture sequences. These target capture sequences are capable of hybridizing to (respective) target nucleic acid sequences from a biological specimen. In some cases, the nucleic acid probes (attached to the polymeric hydrogel 32) include a capture sequence that is common to all or a subset of the probes attached to the polymeric hydrogel 32. As an example, the nucleic acid probes can have a poly A sequence or a poly T sequence. Such probes or amplicons thereof can hybridize to mRNA molecules, cDNA molecules, genomic DNA (gDNA), ribosomal RNA (rRNA), or transfer RNA (tRNA). In this example, the primers 42A, 42B (i.e., the nucleic acid probes) interact with target nucleic acids during an indexing operation. The target nucleic acid(s) interact with the nucleic acid probes that are proximal to a region of the specimen from which the target nucleic acid(s) was/were released. A target-probe hybrid complex can form where the target nucleic acid(s) encounter(s) a complementary target capture sequence on a nucleic acid probe. The location of this target-probe hybrid complex will generally correlate with the region of the biological specimen from which the target nucleic acid(s) was/were derived. It is to be understood that the primers 42A, 42B may be used as a variety of nucleic acid probes, where a plurality of target-probe hybrid complexes is formed from the probes and from the target nucleic acids. The sequences of the target nucleic acids and their location(s) on the substrate 14, 16 will provide spatial genomic information about the nucleic acid content of the biological specimen.

In another example, the primers 42A, 42B are amplification oligonucleotides. In this example, the amplification oligonucleotides can be immobilized to the polymeric hydrogel 32 by single point covalent attachment at or near the 5′ end of the primers 42A, 42B. Any suitable covalent attachment may be used for this purpose. This attachment leaves i) an adapter-specific portion of the primers 42A, 42B free to anneal to its cognate sequencing-ready nucleic acid fragment and ii) the 3′ hydroxyl group of the primers 42A, 42B free/available for nucleotide extension. By “adapter-specific,” it is meant that the individual nucleotide(s) that make up each of the primers 42A, 42B at or near the 3′ end are selected to be complementary to desired base pairs, such as base pairs that make up adapter sequences attached to molecules (e.g., library templates) being amplified.

In examples in which different primers 42A, 42B are used as part of a set, the primers 42A, 42B may be used in sequential paired end sequencing. As examples, the primers 42A, 42B may respectively include P5 and P7 primers, P15 and P7 primers, or any combination of the PA primers, the PB primers, the PC primers, and the PD primers set forth hereinbelow. As further examples, the primers 42A, 42B may respectively include any two PA, PB, PC, and PD primers, or any combination of one PA primer and one PB, PC, or PD primer, or any combination of one PB primer and one PC or PD primer, or any combination of one PC primer and one PD primer.

Specific examples of suitable primers for the different primers 42A, 42B include P5 and P7 primers used on the surface of commercial flow cells sold by Illumina Inc. for sequencing on HISEQ™, HISEQX™, MISEQ™, MISEQDX™ MINISEQ™, NEXTSEQ™, NEXTSEQDX™, NOVASEQ™, NOVASEQX™, GENOME ANALYZER™, ISEQ™, and other instrument platforms.

The P5 primer (which may be a cleavable primer due to the cleavable nucleobase uracil or “n”) is:

P5 #1: 5′ → 3′
(SEQ. ID. NO. 1)
AATGATACGGCGACCACCGAGAUCTACAC;
P5 #2: 5′ → 3′
(SEQ. ID. NO. 2)
AATGATACGGCGACCACCGAGAnCTACAC;
where “n” is inosine in SEQ. ID. NO. 2
or
P5 #3: 5′ → 3′
(SEQ. ID. NO. 3)
AATGATACGGCGACCACCGAGAnCTACAC.
where “n” is alkene-thymidine (i.e., alkene-dT)
in SEQ. ID. NO. 3

The P7 primer (which may be a cleavable primer) may be any of the following:

P7 #1: 5′ → 3′
(SEQ. ID. NO. 4)
CAAGCAGAAGACGGCATACGAnAT;
where “n” is 8-oxoguanine in SEQ. ID. NO. 4
P7 #2: 5′ → 3′
(SEQ. ID. NO. 5)
CAAGCAGAAGACGGCATACnAGAT;
where “n” is 8-oxoguanine in SEQ. ID. NO. 5
P7 #3: 5′ → 3′
(SEQ. ID. NO. 6)
CAAGCAGAAGACGGCATACnAnAT;
where both instances of “n” are 8-oxoguanine
in SEQ. ID. NO. 6
P7 #4: 5′ → 3′
(SEQ. ID. NO. 7)
CAAGCAGAAGACGGCATACGAUAT;
or
P7 #5: 5′ → 3′
(SEQ. ID. NO. 8)
CAAGCAGAAGACGGCATACUAGAT.

The P15 primer (shown as a cleavable primer) is:

P15: 5′ → 3′
(SEQ. ID. NO. 9)
AATGATACGGCGACCACCGAGAnCTACAC.
where “n” is allyl-T (i.e., a thymine nucleotide
analog having an allyl functionality)

The other primers (PA-PD, shown as non-cleavable primers) mentioned above include:

PA 5′ → 3′
(SEQ. ID. NO. 10)
GCTGGCACGTCCGAACGCTTCGTTAATCCGTTGAG;
PB 5′ → 3′
(SEQ. ID. NO. 11)
CGTCGTCTGCCATGGCGCTTCGGTGGATATGAACT;
PC 5′ → 3′
(SEQ. ID. NO. 12)
ACGGCCGCTAATATCAACGCGTCGAATCCGCAACT;
and
PD 5′ → 3′
(SEQ. ID. NO. 13)
GCCGCGTTACGTTAGCCGGACTATTCGATGCAGC.

While not shown in the example sequences for PA-PD, it is to be understood that any of these primers may include a cleavage site, such as uracil, 8-oxoguanine, allyl-T, etc. at any point in the strand. It is to be further understood that the cleavage sites of the primers 42A, 42B that make up a primer set are orthogonal, i.e., the cleavage site of one of the primers 42A, 42B is not susceptible to a cleaving agent used to remove the cleavage site of the other of the primers 42A, 42B, and vice versa.

Each of the amplification primers 42A, 42B disclosed herein may also include a polyT sequence at or near the 5′ end of the primer sequence. In some examples, the polyT region includes from 2 T bases to 20 T bases. As specific examples, the polyT region may include 3, 4, 5, 6, 7, or 10 T bases.

The primers 42A, 42B may alco include a 5′ end functional group that can attach the primer to the polymeric hydrogel 32.

Additional examples of the flow cell 10 are shown in FIG. 2A through FIG. 2D. Each of these example flow cells 10 includes a patterned structure having a plurality of depressions 36 defined in an individual active region 38, where the depressions 36 in each individual active region 38 are separated from immediately adjacent depressions 36 by interstitial regions 37.

In the example shown in FIG. 2A, the first substrate 14, 16 is attached to the lid 22 via the interposer 30 and the bonding material(s) 26A, 26B, 26C, and/or 28. As shown in the figure, the bonding material 26A, 26B, 26C, or 28 is in contact with a first side S₁ of the interposer 30 and attaches the interposer 30 to the substrate 14, 16 (at the first bonding region(s) 40). As further shown in the figure, the bonding material 26A, 26B, 26C, or 28 is in contact with a second side S₂ of the interposer 30 (that is opposed to the first side S₁) and attaches the interposer 30 (and thus the substrate 14, 16 attached to the first side S₁ of the interposer 30) to the lid 22 (at the second bonding region(s) 40′).

Various combinations of the light-switchable bonding material 26A, the solvent-responsive bonding material 26B, the heat-responsive bonding material 26C, or the non-switchable bonding material 28 may be used in this example of the flow cell 10. In an example, the light-switchable bonding material 26A is in contact with one of the first side S₁ or the second side S₂ of the interposer 30, and the non-switchable bonding material 28 is in contact with the other of the second side S₂ or the first side S₁ of the interposer 30. In another example, the solvent-responsive bonding material 26B is in contact with one of the first side S₁ or the second side S₂ of the interposer 30, and the non-switchable bonding material 28 is in contact with the other of the second side S₂ or the first side S₁ of the interposer 30. In yet another example, the heat-responsive bonding material 26C is in contact with one of the first side S₁ or the second side S₂ of the interposer 30, and the non-switchable bonding material 28 is in contact with the other of the second side S₂ or the first side S₁ of the interposer 30. In still other examples, the light-switchable bonding material 26A, the solvent-responsive bonding material 26B, or the heat-responsive bonding material 26C is in contact with both the first side S₁ and the second side S₂ of the interposer 30.

The example flow cell 10 shown in FIG. 2A also includes the plurality of depressions 36 in each active region 38 included therein. Each of the depressions 36 in the active region(s) 38 includes the polymeric hydrogel 32, which has primers 42A, 42B attached thereto (such that the primers 42A, 42B are positioned within the depressions 36). As shown in the figure, the interstitial regions 37 may be free of surface chemistry (i.e., may be devoid/free of polymeric hydrogel 32 and primers 42A, 42B). While FIG. 2A depicts a flow cell 10 including two individual active regions 38, each active region 38 including three depressions 36, it is to be understood that this example of the flow cell 10 may include any number of active regions 38, with each individual active region 38 including any desired number of depressions 36 defined therein (e.g., tens, hundreds, thousands, or millions of depressions 36 within an individual active region 38).

In the example shown in FIG. 2B, the first substrate 14, 16 is also attached to the lid 22 via the interposer 30 and the bonding material(s) 26A, 26B, 26C, and/or 28. In this example, however, the bonding region(s) 40 include(s) a plurality of depressions 36′ defined in the first substrate 14, 16. The depressions 36′ that are defined in the first substrate 14, 16 at the (first) bonding region(s) 40 may have the same or different dimensions as the depressions 36 that are defined in the active region(s) 38 of the substrate 14, 16 (these dimensions will be described) and the depressions 36′ may be separated by interstitial regions 37′. The depressions 36′ in the bonding region(s) 40 of the substrate 14, 16 include the bonding material 26A, 26B, 26C, or 28 therein, such that the bonding material 26A, 26B, 26C, or 28 is in contact with the first side S₁ of the interposer 30 and attaches the interposer 30 to the first substrate 14, 16 (at the first bonding region(s) 40). As further shown in the figure, the bonding material 26A, 26B, 26C, or 28 is in contact with a second side S₂ of the interposer 30 (the second side S₂ being opposed to the first side S₁) and attaches the interposer 30 (and thus the substrate 14, 16, which is attached to the first side S₁ of the interposer 30) to the lid 22 at the second bonding region(s) 40′.

Various combinations of the light-switchable bonding material 26A, the solvent-responsive bonding material 26B, the heat-responsive bonding material 26C, or the non-switchable bonding material 28 may be used in this example of the flow cell 10. In an example, the light-switchable bonding material 26A is in contact with one of the first side S₁ or the second side S₂ of the interposer 30, and the non-switchable bonding material 28 is in contact with the other of the second side S₂ or the first side S₁ of the interposer 30. In another example, the solvent-responsive bonding material 26B is in contact with one of the first side S₁ or the second side S₂ of the interposer 30, and the non-switchable bonding material 28 is in contact with the other of the second side S₂ or the first side S₁ of the interposer 30. In still another example, the heat-responsive bonding material 26C is in contact with one of the first side S₁ or the second side S₂ of the interposer 30, and the non-switchable bonding material 28 is in contact with the other of the second side S₂ or the first side S₁ of the interposer 30. In yet other examples, the light-switchable bonding material 26A the solvent-responsive bonding material 26B, or the heat-responsive bonding material 26C is in contact with both the first side S₁ and the second side S₂ of the interposer 30.

The example flow cell 10 shown in FIG. 2B also includes the plurality of depressions 36 in each active region 38 included therein. Unlike the depressions 36′ in the bonding region(s) 40, the depressions 36 within the active region(s) 38 do not have any bonding material 26A, 26B, 26C, or 28 disposed therein. Rather, the depressions 36 in the active region(s) 38 include the polymeric hydrogel 32 and the primers 42A, 42B attached to the polymeric hydrogel 32. While FIG. 2B depicts two active regions 38, each including three depressions 36, it is to be understood that this example of the flow cell 10 may include any number of active regions 38, with each individual active region 38 including any desired number of depressions 36 (e.g., tens, hundreds, thousands, or millions of depressions 36 within an individual active region 38).

In the example shown in FIG. 2C, rather than being attached to the lid 22, the first substrate 14, 16 is attached to the second substrate 24 via the interposer 30 and the bonding material(s) 26A, 26B, 26C, and/or 28. In this example, the (first) bonding region(s) 40 of the substrate 14, 16 include(s) a plurality of depressions 36′ defined therein, and the (second) bonding region(s) 40′ of the second substrate 24 also include(s) a plurality of depressions 36′ defined therein. The depressions 36′ in the first and second bonding region(s) 40, 40′ may have the same or different dimensions as the depressions 36 that are defined in the active region(s) 38 (these dimensions will be described). The depressions 36′ in the first bonding region(s) 40 of the substrate 14, 16 include the bonding material 26A, 26B, 26C, or 28 therein, such that the bonding material 26A, 26B, 26C, or 28 is in contact with the first side S₁ of the interposer 30 and attaches the interposer 30 to the first substrate 14, 16 (at the first bonding region(s) 40). The depressions 36′ in the second bonding region(s) 40′ of the second substrate 24 also include the bonding material 26A, 26B, 26C, or 28 therein, such that the bonding material 26A, 26B, 26C, or 28 is in contact with the second side S₂ of the interposer 30 and attaches the interposer 30 (and thus the substrate 14, 16) to the second substrate 24 (at the second bonding region(s) 40′).

Various combinations of the light-switchable bonding material 26A, the solvent-responsive bonding material 26B, the heat-responsive bonding material 26C, and/or the non-switchable bonding material 28 may be used in this example of the flow cell 10. In an example, the light-switchable bonding material 26A is in contact with one of the first side S₁ or the second side S₂ of the interposer 30, and the non-switchable bonding material 28 is in contact with the other of the second side S₂ or the first side S₁ of the interposer 30. As another example, the solvent-responsive bonding material 26B can be in contact with one of the first side S₁ or the second side S₂ of the interposer 30, and the non-switchable bonding material 28 can be in contact with the other of the second side S₂ or the first side S₁ of the interposer 30. In still another example, the heat-responsive bonding material 26C is in contact with one of the first side S₁ or the second side S₂ of the interposer 30, and the non-switchable bonding material 28 is in contact with the other of the second side S₂ or the first side S₁ of the interposer 30. In yet other examples, the light-switchable bonding material 26A, the solvent-responsive bonding material 26B, or the heat-responsive bonding material 26C is in contact with both the first side S₁ and the second side S₂ of the interposer 30.

The example flow cell 10 shown in FIG. 2C also includes the plurality of depressions 36 in each active region 38, 38′ included therein. In particular, the depressions 36 in the first substrate 14, 16 form the first active regions 38, and the depressions 36 in the second substrate 24 form the second active regions 38′. Each of the depressions 36 in the first and second active region(s) 38, 38′ includes a polymeric hydrogel 32, which has primers 42A, 42B attached thereto. As such, in an example, the second substrate 24 is attached to the second side S₂ (of the interposer 30), the second substrate 24 includes a second active region 38′ that is spatially separate from a second bonding region 40′, and the polymeric hydrogel 32 positioned at the second active region 38′.

While FIG. 2C depicts a flow cell 10 including two active regions 38 (of the first substrate 14, 16) and two active regions 38′ (of the second substrate 24), each active region 38, 38′ including three depressions 36, it is to be understood that this example of the flow cell 10 may include any number of active regions 38, 38′, with each individual active region 38, 38′ including any desired number of depressions 36 defined therein (e.g., tens, hundreds, thousands, or millions of depressions 36 within an individual active region 38, 38′).

In the example shown in FIG. 2D, the first substrate 14, 16 is attached to the second substrate 24 via the interposer 30 and the bonding material(s) 26A, 26B, 26C, and/or 28. In this example, the (first) bonding region(s) 40 of the substrate 14, 16 include(s) a plurality of depressions 36′ defined therein, whereas there are no depressions 36′ defined in the (second) bonding region(s) 40′ of the second substrate 24. The depressions 36′ in the first bonding region(s) 40 of the substrate 14, 16 may have the same or different dimensions as the depressions 36 that are defined in the active region(s) 38 (these dimensions will be described). The depressions 36′ in the first bonding region(s) 40 of the substrate 14, 16 include the bonding material 26A, 26B, 26C, or 28 therein, such that the bonding material 26A, 26B, 26C, or 28 is in contact with the first side of the interposer 30 and attaches the interposer 30 to the first substrate 14, 16 (at the bonding region(s) 40).

Various combinations of the light-switchable bonding material 26A, the solvent-responsive bonding material 26B, or the non-switchable bonding material 28 may be used in this example of the flow cell 10. In an example, the light-switchable bonding material 26A is in contact with one of the first side S₁ or the second side S₂ of the interposer 30, and the non-switchable bonding material 28 is in contact with the other of the second side S₂ or the first side S₁ of the interposer 30. In another example, the solvent-responsive bonding material 26B is in contact with one of the first side S₁ or the second side S₂ of the interposer 30, and the non-switchable bonding material 28 is in contact with the other of the second side S₂ or the first side S₁ of the interposer 30. In still another example, the heat-responsive bonding material 26C is in contact with one of the first side S₁ or the second side S₂ of the interposer 30, and the non-switchable bonding material 28 is in contact with the other of the second side S₂ or the first side S₁ of the interposer 30. In yet other examples, the light-switchable bonding material 26A, the solvent-responsive bonding material 26B, or the heat-responsive bonding material 26C is in contact with both the first side S₁ and the second side S₂ of the interposer 30.

The example flow cell 10 shown in FIG. 2D also includes the plurality of depressions 36 in each active region 38, 38′ included therein. Each of the depressions 36 in the active region(s) 38, 38′ includes a polymeric hydrogel 32, which has primers 42A, 42B attached thereto. While FIG. 2D depicts a flow cell 10 including two active regions 38 (of the first substrate 14, 16) and two active regions 38′ (of the second substrate 24), each including three depressions 36, it is to be understood that this example of the flow cell 10 may include any number of active regions 38, 38′, with each individual active region 38, 38′ including any desired number of depressions 36 defined therein (e.g., tens, hundreds, thousands, or millions of depressions 36 within an individual active region 38, 38′).

Without being bound by any particular theory, it is believed that the depressions 36′ in the bonding region(s) 40, 40′, when included, facilitate enhanced attachment of the substrate 14, 16 to the lid 22 or to the second substrate 24 due, at least in part, to the additional surface area that is available for bonding.

In any of the examples shown in FIG. 2A through FIG. 2D, different layouts of the depressions 36 and interstitial regions 37 within the individual active regions 38 and different layouts of the depressions 36′ and interstitial regions 37′ in the first and second bonding region(s) 40, 40′ may be envisaged, including regular, repeating, and non-regular patterns. In an example, the depressions 36, 36′ are disposed in a hexagonal grid for close packing and improved density. Other layouts of the depressions 36, 36′ may include, for example, rectilinear (rectangular) layouts, triangular layouts, and so forth. In some examples, the layout or pattern can be an x-y format in rows and columns. In other examples, the layout or pattern can be a repeating arrangement of the depressions 36 and the interstitial regions 37 or of the depressions 36′ and the interstitial regions 37′. In still other examples, the layout or pattern can be a random arrangement of the depressions 36 and the interstitial regions 37 or of the depressions 36′ and the interstitial regions 37′.

The layout or pattern of the depressions 36, 36′ may be characterized with respect to the density (number) of the depressions 36, 36′ in a defined area (e.g., within a defined active region 38, 38′ or within a defined bonding region 40, 40′). For example, the depressions 36, 36′ may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high-density array may be characterized as having the depressions 36, 36′ separated by less than about 100 nm, a medium-density array may be characterized as having the depressions 36, 36′ separated by about 400 nm to about 1 μm, and a low-density array may be characterized as having the depressions 36 separated by greater than about 1 μm.

The layout or pattern of the depressions 36, 36′ may also or alternatively be characterized in terms of the average pitch, or the spacing from the center of one depression 36, 36′ to the center of an immediately adjacent depression 36, 36′. Alternatively, the average pitch may refer to the spacing from a left edge of one depression 36, 36′ to the left edge of an immediately adjacent depression 36, 36′. As an additional alternative, the average pitch may refer to the spacing from the right edge of one depression 36, 36′ to the right edge of an immediately adjacent depression 36, 36′. The pattern can be regular, such that the coefficient of variation around the average pitch is small, or the pattern can be non-regular in which case the coefficient of variation can be relatively large. In either case, the average pitch can be, for example, about 50 nm, about 0.15 μm, about 0.5 μm, about 1 μm, about 5 μm, about 10 μm, about 100 μm, or more or less. The average pitch for a particular pattern of depressions 36, 36′ can be between one of the lower values and one of the upper values selected from the ranges herein.

The size of each of the depressions 36, 36′ may be characterized by the volume, opening area, depth, and/or diameter or length and width of the depressions 36, 36′. For example, the volume can range from about 1×10⁻³ μm³ to about 100 μm³, e.g., about 1×10⁻² μm³, about 0.1 μm³, about 1 μm³, about 10 μm³, or more, or less. For another example, the opening area can range from about 1×10⁻³ μm² to about 100 μm², e.g., about 1×10⁻² μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

It is to be understood that the different layouts and pitches set forth herein for the depressions 36, 36′ may also be used for the functionalized pads 27 shown in FIG. 1C. Unlike the depressions 36, 36′, however, the size of each of the functionalized pads 27 may be characterized by the volume or by the length and width of the functionalized pads 27. For example, the volume can range from about 1×10⁻³ μm³ to about 100 μm³, e.g., about 1×10⁻² μm³, about 0.1 μm³, about 1 μm³, about 10 μm³, or more, or less. As another example, the diameter or each of the length and width can range from about 0.1 μm to about 100 μm, e.g., about 0.5 μm, about 1 μm, about 10 μm, or more, or less.

Various methods of using the bonding materials 26A, 26B, 26C, and 28 as part of a process of forming the flow cell 10 will now be described.

### Method of Forming the Flow Cell

Various examples of a method that may be used as part of a process of generating the flow cell(s) 10 described herein are depicted in FIG. 3A through FIG. 3C.

Some examples of the method generally involve applying a polymeric hydrogel 32 to a first region of a first substrate 14, 16, thereby forming an active region 38 that is spatially separate from a bonding region 40 of the first substrate 14, 16; applying a light-switchable bonding material 26A to i) the bonding region 40 or ii) at least one of a first side S₁ or a second side S₂ of an interposer 30, wherein the first side S₁ is opposed to the second side S₂; attaching the first side S₁ of the interposer 30 to the bonding region 40; and attaching a lid 22 or a second substrate 24 to the second side S₂ of the interposer 30.

Other examples of the method generally involve applying a polymeric hydrogel 32 to a first region of a first substrate 14, 16, thereby forming an active region 38 that is spatially separate from a bonding region 40 of the first substrate 14, 16; applying a solvent-responsive bonding material 26B to i) the bonding region 40 or ii) at least one of a first side S₁ or a second side S₂ of an interposer 30, wherein the first side S₁ is opposed to the second side S₂; attaching the first side S₁ of the interposer 30 to the bonding region 40; and attaching a lid 22 or a second substrate 24 to the second side S₂ of the interposer 30.

Further examples of the method generally involve applying a polymeric hydrogel 32 to a first region of a first substrate 14, 16, thereby forming an active region 38 that is spatially separate from a bonding region 40 of the first substrate 14, 16; applying a heat-responsive bonding material 26C to i) the bonding region 40 or ii) at least one of a first side S₁ or a second side S₂ of an interposer 30, wherein the first side S₁ is opposed to the second side S₂; attaching the first side S₁ of the interposer 30 to the bonding region 40; and attaching a lid 22 or a second substrate 24 to the second side S₂ of the interposer 30.

As shown in FIG. 3A, the substrate 14 (or the layer 20 of the substrate 16) has a lane 34 defined therein, where the lane 34 includes the polymeric hydrogel 32 having primers 42A, 42B attached thereto (similar to the unpatterned structure including the lane 34 depicted in FIG. 1B). It is to be understood, however, that the examples of the method shown in FIG. 3A through FIG. 3C may alternatively utilize a substrate 14, 16 having depressions 36 defined therein (similar to the patterned structures shown in FIG. 2A through FIG. 2D), or a substrate 14, 16 having functionalized pads 27 defined thereon (similar to the patterned structure shown in FIG. 1C). In any case, either the single layer substrate 14 or the multi-layer substrate 16 including the base support 18 and the layer 20 may be used in these examples of the method.

The lane 34, when included, may be defined in the substrate 14 (or in the layer 20 of the substrate 16) using any suitable technique (e.g., etching, nanoimprint lithography, photolithography, etc.). The patterning technique that is used for the substrate 14 or the layer 20 will depend, in part, upon the material selected for the substrate 14 or the layer 20 and upon the desired features of the lane 34 (e.g., in terms of concavity). As an example of forming the lane 34, when the substrate 14 or the layer 20 includes a resin material, a working stamp (including a negative replica of the lane 34) may be pressed into the resin material of the substrate 14 or layer 20 while the resin is soft. The resin of the substrate 14 or layer 20 may then be cured while the working stamp is in place, e.g., via exposure to actinic radiation or to heat. After curing, the working stamp is released, which forms the lane 34 in the substrate 14 or in the layer 20.

While not shown in FIG. 3A through FIG. 3C, the depressions 36, when included, may be defined in the substrate 14 (or in the layer 20 of the substrate 16) using any suitable technique (e.g., etching, nanoimprint lithography, photolithography, etc.). The patterning technique that is used to define the depressions 36 in the substrate 14 or the layer 20 will depend, in part, upon the material selected for the substrate 14 or the layer 20. As an example of forming the depressions 36, when the substrate 14 or the layer 20 includes a resin material, a working stamp (including a negative replica of the depressions 36) may be pressed into the resin material of the substrate 14 or layer 20 while the resin is soft. The resin of the substrate 14 or layer 20 may then be cured while the working stamp is in place, e.g., via exposure to actinic radiation or to heat. After curing, the working stamp is released, which forms the depressions 36 in the active region 38 of the substrate 14, 16. It is to be understood that in examples in which depressions 36′ are included in (a) bonding region(s) 40 of the substrate 14, 16, the depressions 36′ may be formed using the same or a different patterning technique. In some instances, the depressions 36 in the active region(s) 38 and the depressions 36′ in the bonding region(s) 40 are formed simultaneously.

After the desired feature has been formed in the substrate 14, 16, such as the lane 34 or the depressions 36, the polymeric hydrogel 32 is deposited within the lane 34 (as shown in FIG. 3A), or within the depressions 36 (not shown), or to form the functionalized pads 27 (not shown). The polymeric hydrogel 32 may be any suitable example disclosed herein and may be deposited using any suitable deposition technique disclosed herein. In a specific example, the polymeric hydrogel 32 is PAZAM.

Selective deposition of the polymeric hydrogel 32 may be performed such that the polymeric hydrogel 32 is deposited at desired areas of the substrate 14, 16 (e.g., where it is desirable to form an active region(s) 38) and such that the polymeric hydrogel 32 is excluded from other areas of the substrate 14, 16 (e.g., such as the bonding region(s) 40 of the substrate 14, 16 and/or from the interstitial regions 37). This selective deposition may be facilitated using masking techniques (e.g., using photolithography or a sacrificial layer) or using blanket deposition followed by a removal step, such as a polishing process. When the bonding region 40 includes depressions 36′ (not shown in FIG. 3A), it is to be understood that the blanket deposition process is one in which the polymeric hydrogel 32 is deposited at the active region 38 and not at the bonding region 40. An example of such a blanket deposition process utilizes a precision gantry tool. In this example, polishing may be used to remove the polymeric hydrogel 32 from the interstitial regions 37 (e.g., when the active region 38 includes depressions 36). When the bonding region 40 does not include depressions 36′, it is to be understood that the blanket deposition process may alternatively include one in which the polymeric hydrogel 32 is deposited at the active region 38 and at the bonding region 40. In this example, the polymeric hydrogel 32 is polished from both the bonding region 40 and the interstitial regions 37.

In any of these examples, the polishing process may involve using a chemical slurry (including, e.g., an abrasive, a buffer, a chelating agent, a surfactant, and/or a dispersant) that can remove the polymeric hydrogel 32 from desired regions of the substrate 14, 16 without deleteriously affecting the underlying substrate 14, 16 and without deleteriously affecting the polymeric hydrogel 32 that is to be left intact within the active region(s) 38. Polishing may also be performed with a solution that does not include the abrasive particles. The polishing process may also be performed using (a) polishing head(s)/pad(s) or (an)other polishing tool(s). As an example, the polishing head may be a Strasbaugh ViPRR II polishing head.

Some examples of the method further involve activating portions of the substrate 14, 16 (where it is desirable to form (an) active region(s) 38) prior to depositing the polymeric hydrogel 32 thereon. Activation of the substrate 14, 16 may facilitate attachment of the polymeric hydrogel 32 thereto and may be involve silanization or plasma ashing.

Plasma ashing involves the generation of —OH groups at a surface via exposure of the surface to oxygen plasma.

Silanization involves the application of a silane or silane derivative over the surface of the substrate 14 or the layer 20 (of the substrate 16). The selection of the silane or silane derivative may depend, in part, upon the polymeric hydrogel 26 that is to be applied. Some example silane derivatives include a cycloalkene unsaturated moiety, such as norbornene, a norbornene derivative (e.g., a (hetero)norbornene including an oxygen or nitrogen in place of one of the carbon atoms), trans-cyclooctene, trans-cyclooctene derivatives, trans-cyclopentene, trans-ycloheptene, trans-cyclononene, bicyclo[3.3.1]non-1-ene, bicyclo[4.3.1]dec-1 (9)-ene, bicyclo[4.2.1]non-1(8)-ene, and bicyclo[4.2.1]non-1-ene. Any of these cycloalkenes can be substituted, for example, with an R group, such as hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An example of the norbornene derivative includes [(5-bicyclo[2.2.1]hept-2-enyl)ethyl]trimethoxysilane. Other example silane derivatives include a cycloalkyne unsaturated moiety, such as cyclooctyne, a cyclooctyne derivative, or bicyclononynes (e.g., bicyclo[6.1.0]non-4-yne or derivatives thereof, bicyclo[6.1.0]non-2-yne, or bicyclo[6.1.0]non-3-yne). These cycloalkynes can be substituted with any of the R groups described herein for the cycloalkene. The method used to apply the silane or silane derivative may vary depending upon the silane or silane derivative that is being used. Examples of suitable silanization methods include vapor deposition (e.g., a YES method), spin coating, or other deposition methods.

The functionalized pads 27, when included, can be formed on a surface of the substrate 14 or of the layer 20 using any suitable patterning technique disclosed herein. In some instances, masking techniques may be used to form the functionalized pads 27. With masking techniques, a sacrificial layer and/or photoresist may be used to block areas of the substrate 14, 16 that are not to receive the polymeric hydrogel 32 that define the functionalized pads 27. After the deposited polymeric hydrogel 32 is cured, the sacrificial layer and/or photoresist may be removed, which exposes the underlying interstitial regions 37 and the physically isolated functionalized pads 27.

In some examples, the polymeric hydrogel 32 that is deposited at the active region(s) 38 does not have primers 42A, 42B pre-grafted thereto (and thus is a non-pre-grafted polymeric hydrogel 32). In these examples, the primers 42A, 42B are grafted to the polymeric hydrogel 32 after the polymeric hydrogel 32 has been applied within the first region of the substrate 14, 16 (to form the active region 38). Thus, in examples, the method further includes attaching a plurality of primers 42A, 42B to the polymeric hydrogel 32.

Grafting of the primers 42A, 42B to the non-pre-grafted polymeric hydrogel 32 may be performed using a primer grafting solution or mixture that includes the primers 42A, 42B, water, a buffer, and a catalyst. The deposition of the primer grafting solution or mixture over the polymeric hydrogel 32 may involve dunk coating, spray coating, puddle dispensing, or any other suitable technique that will attach the primers 42A, 42B to portions of the polymeric hydrogel 32 where formation of an active region 38 is desired.

Dunk coating may involve submerging the substrate 14, 16 (having the polymeric hydrogel 32 in a lane 34, or in the form of functionalized pads 27, or in depressions 36) into a series of temperature-controlled baths. The baths may also be flow controlled and/or covered with a nitrogen blanket. The baths may include the primer grafting solution or mixture. Throughout the various baths, the primers 42A, 42B will attach to the polymeric hydrogel 32. In an example, substrate 14, 16 will be introduced into a first bath including the primer solution or mixture where a reaction takes place to attach the primers 42A, 42B and then the substrate 14, 16 will be moved to additional baths for washing. The substrate 14, 16 may be moved from bath to bath with a robotic arm or manually. A drying system may also be used in dunk coating.

Spray coating may be accomplished by spraying the primer grafting solution or mixture directly onto the polymeric hydrogel 32 positioned on the substrate 14, 16. The spray coated substrate 14, 16 may be incubated for a time ranging from about 4 minutes to about 60 minutes at a temperature ranging from about 0° C. to about 70° C. After incubation, the primer grafting solution or mixture may be diluted and removed using, for example, a spin coater.

Puddle dispensing may be performed according to a pool and spin off method, and thus may be accomplished with a spin coater. The primer grafting solution or mixture may be applied (manually or via an automated process) to the polymeric hydrogel 32 positioned on the substrate 14, 16. The applied primer solution or mixture may be applied to or spread across the entire hydrogel-coated surface of the substrate 14, 16. The substrate 14, 16 may be incubated for a time ranging from about 2 minutes to about 60 minutes at a temperature ranging from about 0° C. to about 80° C. to achieve primer grafting. After incubation, the primer grafting solution or mixture (including any ungrafted primers) may be diluted and removed using, for example, the spin coater.

Grafting of the primers 42A, 42B to the polymeric hydrogel 32 may also be accomplished using a flow through process, which takes place after the lid 22 or the second substrate 24 has been bonded. In the flow through process, the primer grafting solution or mixture may be introduced to the substrate 14, 16 of the flow cell 10 through an input port and may be maintained at desired regions of the substrate 14, 16 for a time sufficient (i.e., an incubation period) for the primers 42A, 42B to attach to the polymeric hydrogel 32. The primer grafting solution or mixture may then be removed via an output port. After primer attachment, additional fluid(s) may be used to wash the substrate 14, 16 having the polymeric hydrogel 32 and the primers 42A, 42B thereon.

In other instances, the polymeric hydrogel 32 that is deposited at the active region(s) 38 is a “pre-grafted” polymeric hydrogel 32, meaning that the polymeric hydrogel 32 has the plurality of primers 42A, 42B attached/grafted thereto before the hydrogel 32 is applied to the substrate 14, 16. As such, in these instances, the attaching of the primers 42A, 42B to the polymeric hydrogel 32 is performed prior to applying the polymeric hydrogel 32 over the first portion of the substrate 14, 16 (and thus prior to forming the active region(s) 38). The primers 42A, 42B may be pre-grafted to the polymeric hydrogel 32 (to form the pre-grafted polymeric hydrogel 32) using any suitable grafting technique. As an example, a primer solution or mixture may be formed, where the primer solution or mixture includes the primer(s) 42A, 42B, the polymeric hydrogel 32, water, a buffer, and a catalyst. Functional groups at or near the 5′ end of the primers 42A, 42B in the primer solution or mixture react with reactive surface functional groups of the polymeric hydrogel 32 and become attached thereto, thereby forming the pre-grafted polymeric hydrogel 32.

Regardless of whether the pre-grafted polymeric hydrogel 32 or the non-pre-grafted polymeric hydrogel 32 is used in the method, the primers 42A, 42B may include any example of the P5, P7, P15, and PA-PD primers 42A, 42B disclosed herein and may form a primer set, as described herein.

Turning now to FIG. 3B, after the polymeric hydrogel 32 has been applied within the active region(s) 38 (and after the primers 42A, 42B have been grafted thereto, when the non-pre-grafted polymeric hydrogel 32 is used), the method proceeds by attaching an interposer 30 to the (first) bonding region(s) 40 of the substrate 14, 16. The interposer 30 may include any suitable material disclosed herein.

In some instances, the attaching of the interposer 30 to the first bonding region(s) 40 of the substrate 14, 16 involves applying the bonding material 26A, 26B, 26C, or 28 to a first side S₁ of the interposer 30 and attaching the first side S₁ of the interposer 30 to the first bonding region(s) 40. In other instances, the attaching of the interposer 30 to the first bonding region(s) 40 involves applying the bonding material 26A, 26B, 26C, or 28 to the first bonding region(s) 40 (e.g., to the substrate 14, 16 at the first bonding region(s) 40) and attaching the first side S₁ of the interposer 30 to the substrate 14, 16 at the first bonding region(s) 40. In any of these instances, the attachment of the interposer 30 to the first bonding region(s) 40 of the substrate 14, 16 generates the structure depicted in FIG. 3B. Further, in any of these instances, the interposer 30 may be attached to the substrate 14, 16 at the bonding region(s) 40 using the light-switchable bonding material 26A, or using the solvent-responsive bonding material 26B, using the heat-responsive bonding material 26C, or using the non-switchable bonding material 28.

The light-switchable bonding material 26A, when used to attach the interposer 30 to the substrate 14, 16 at the first bonding region(s) 40, may be any suitable example disclosed herein. Further, in an example, after the interposer 30 is attached to the substrate 14, 16 at the first bonding region 40 (using the intervening light-switchable bonding material 26A), the method involves exposing the light-switchable bonding material 26A to a preselected light wavelength to increase properties of adhesion within the light-switchable bonding material 26A. This preselected light wavelength may be in the ultraviolet (UV) range or in the visible light range. As such, in some examples of the method, the light-switchable bonding material 26A exhibits improved adhesion when exposed to UV light and exhibits reduced adhesion when exposed to visible light; and the method further comprises placing the first side S₁ (of the interposer 30) and the bonding region 40 in contact with each other after the light-switchable bonding material 26A is applied, and then exposing the light-switchable bonding material 26A to UV light. It is to be understood that the light exposure may take place at any desired angle with respective to the interface where the light-switchable bonding material 26A is present. If the light is directed through the substrate 14, 16 or through the interposer 30, such components 14, 16 or 30 may be transparent to the wavelength of light being used. The duration of light exposure may depend, in part, upon the thickness of the light-switchable bonding material 26A, the thickness of the lid 22 or the second substrate 24, and the light dosage (mJ/cm²) used.

The solvent-responsive bonding material 26B, when used to attach the interposer 30 to the substrate 14, 16 at the first bonding region 40, may be any suitable hydrogel-based or non-hydrogel-based example disclosed herein. In at least some of these examples, the solvent-responsive bonding material 26B is a hydrogel adhesive. Further, in an example, after the interposer 30 is attached to the substrate 14, 16 at the first bonding region 40 (using the intervening solvent-responsive bonding material 26B), the method involves exposing the solvent-responsive bonding material 26B to drying, curing, or heating conditions to increase properties of adhesion within the solvent-responsive bonding material 26B.

The heat-responsive bonding material 26C, when used to attach the interposer 30 to the substrate 14, 16 at the first bonding region 40, may be any suitable example disclosed herein. Further, in an example, after the interposer 30 is attached to the substrate 14, 16 at the first bonding region 40 (using the intervening heat-responsive bonding material 26C), the method involves exposing the heat-responsive bonding material 26C to drying, curing, or heating conditions to increase properties of adhesion within the heat-responsive bonding material 26C.

The non-switchable bonding material 28, when used to attach the interposer 30 to the substrate 14, 16 at the bonding region 40, may be any suitable example disclosed herein. Further, in an example, after the interposer 30 is attached to the substrate 14, 16 at the bonding region 40 (using the intervening non-switchable bonding material 28), the method involves exposing the non-switchable bonding material 28 to drying, curing, or heating conditions to increase properties of adhesion within the non-switchable bonding material 28.

While not shown in FIG. 3B, in some examples, prior to attaching the first side S₁ of the interposer 30 to the bonding region 40, the method further comprises forming a plurality of depressions 36′ in the bonding region 40, and after attaching the first side S₁ of the interposer 30 to the bonding region 40, the light-switchable bonding material 26A at least partially fills the plurality of depressions 36′ in the bonding region 40. Further, while not shown in FIG. 3B, in other examples, prior to attaching the first side S₁ of the interposer 30 to the bonding region 40, the method further comprises forming a plurality of depressions 36′ in the bonding region 40, and wherein after attaching the first side S₁ of the interposer 30 to the bonding region 40, the solvent-responsive bonding material 26B at least partially fills the plurality of depressions 36′ in the bonding region 40. Still further, while not shown in FIG. 3B, in other examples, prior to attaching the first side S₁ of the interposer 30 to the bonding region(s) 40, the method further comprises forming a plurality of depressions 36′ in the bonding region 40. In these examples, after attaching the first side S₁ of the interposer 30 to the bonding region(s) 40, the heat-responsive bonding material 26C at least partially fills the depressions 36′ in the bonding region(s) 40. Forming depressions 36′ in the bonding region(s) 40 of the substrate 14, 16 can be used to generate a patterned structure similar to that shown in FIG. 2B through FIG. 2D, and can enhance the bonding of the interposer 30 to the substrate 14, 16 at the bonding region(s) 40.

Turning now to FIG. 3C, after the first side S₁ of the interposer 30 has been attached to the substrate 14, 16 at the first bonding region(s) 40, the method proceeds by attaching a lid 22 or a second substrate 24 to the second side S₂ of the interposer 30 (e.g., at (a) second bonding region(s) 40′), where the second side S₂ of the interposer 30 is opposed to the first side S₁. The lid 22 or the second substrate 24 may be any suitable example disclosed herein. The attachment of the lid 22 or the second substrate 24 to the second side S₂ of the interposer 30 forms an enclosed area (e.g., an active region 38 and/or a flow channel 12) of the flow cell 10.

In some instances, the attaching of the lid 22 or the second substrate 24 to the second side S₂ of the interposer 30 at the second bonding region(s) 40′ involves applying the bonding material 26A, 26B, 26C, or 28 to the second side S₂ of the interposer 30 and attaching the second side S₂ of the interposer 30 to the second bonding region(s) 40′. In other instances, the attaching of the second side S₂ of the interposer 30 to the second bonding region(s) 40′ involves applying the bonding material 26A, 26B, 26C, or 28 to the second bonding region(s) 40′ (e.g., to the second bonding region(s) 40′ of the lid 22 or the second substrate 24) and attaching the second side S₂ of the interposer 30 to the lid 22 or substrate 24 at the second bonding region(s) 40′. In any of these instances, the attachment of the second side S₂ of the interposer 30 to the second bonding region(s) 40′ of the lid 22 or the second substrate 24 generates the structure depicted in FIG. 3C. Further, in any of these instances, the second side S₂ of the interposer 30 may be attached to the lid 22 or to the second substrate 24 at the second bonding region(s) 40′ using the light-switchable bonding material 26A, or using the solvent-responsive bonding material 26B, using the heat-responsive bonding material 26C, or using the non-switchable bonding material 28. Pressure may be applied to the lid 22 or to the second substrate 24 to aid in sealing the lid 22 or the second substrate 24 to the second side S₂ of the interposer 30. In an example, the amount of pressure that is applied to the lid 22 or to the second substrate 24 ranges from about 40 psi to about 150 psi.

While not shown in FIG. 3C, the second substrate 24, when used in the method instead of the lid 22, may include its own respective active region(s) 38′, where the active region(s) 38′ are defined by the areas of the substrate 24 that have surface chemistry thereon (similar to the second substrate 24 shown in FIG. 2C and FIG. 2D). As such, in some examples of the method, the second substrate 24 is attached to the second side S₂ of the interposer 30, and the second substrate 24 includes a second active region 38′ that is spatially separate from a second bonding region 40′, and the polymeric hydrogel 32 is positioned at the second active region 38′.

As can be understood from the examples described in reference to FIG. 3B and FIG. 3C, various combinations of the light-switchable bonding materials 26A, the solvent-responsive bonding materials 26B, the heat-responsive bonding materials 26C, or the non-switchable bonding materials 28 disclosed herein may be used on the first and the second side S₁, S₂ of the interposer 30. Some example configurations of the bonding materials 26A, 26B, 26C, 28 will now be described.

In one example, the light-switchable bonding material 26A is applied to the bonding region 40 or to the first side S₁ of the interposer 30 and attaches the first side S₁ of the interposer 30 to the bonding region 40. In this example, the attaching of the lid 22 or the second substrate 24 to the second side S₂ of the interposer 30 involves applying a non-switchable bonding material 28 to the second side S₂; and contacting the lid 22 or the second substrate with the non-switchable bonding material 28. Once the non-switchable bonding material 28 is applied and the components are in contact with one another, curing and/or drying may be performed depending upon the type of non-switchable bonding material 28 that is used.

In another example, the light-switchable bonding material 26A is applied to the second side S₂ of the interposer 30 and attaches the second side S₂ of the interposer 30 to the lid 22 or to the second substrate 24. In this example, the attaching of the first side S₁ of the interposer 30 to the bonding region 40 involves applying the non-switchable bonding material 28 to the first side S₁ or to the bonding region 40 and then placing the first side S₁ and the bonding region 40 in contact with each other. Once the non-switchable bonding material 28 is applied and the components are in contact with one another, curing and/or drying may be performed depending upon the type of non-switchable bonding material 28 that is used.

In still another example, the light-switchable bonding material 26A is applied to both the first side S₁ and the second side S₂ of the interposer 30, attaches the first side S₁ of the interposer 30 to the bonding region 40, and attaches the second side S₂ of the interposer 30 to the lid 22 or to the second substrate 24. Once the light-switchable bonding material 26A is applied and the components are in contact with one another, suitable light exposure is performed to achieve adhesion. In this example, it is to be understood that the material stack at the bonding regions 40, 40′ may be exposed to a single light exposure event to achieve adhesion. Alternatively, in this example, light exposure events may be performed after the light-switchable bonding material 26A is applied and the interposer 30 is in position, and again after the light-switchable bonding material 26A is applied and the lid 22 or the second substrate 24 is in position.

In one example, the solvent-responsive bonding material 26B is applied to the bonding region 40 or to the first side S₁ of the interposer 30 and attaches the first side S₁ of the interposer 30 to the bonding region 40. In this example, the attaching of the lid 22 or the second substrate 24 to the second side S₂ of the interposer 30 involves applying a non-switchable bonding material 28 to the second side S₂; and contacting the lid 22 or the second substrate 24 with the non-switchable bonding material 28. Once the non-switchable bonding material 28 is applied and the components are in contact with one another, curing and/or drying may be performed depending upon the type of non-switchable bonding material 28 that is used.

In another example, the solvent-responsive bonding material 26B is applied to the second side S₂ of the interposer 30 and attaches the second side S₂ of the interposer 30 to the lid 22 or to the second substrate 24. In this example, the attaching of the first side S₁ of the interposer 30 to the bonding region 40 involves applying the non-switchable bonding material 28 to the first side S₁ or to the bonding region 40 and then placing the first side S₁ and the bonding region 40 in contact with each other. Once the non-switchable bonding material 28 is applied and the components are in contact with one another, curing and/or drying may be performed depending upon the type of non-switchable bonding material 28 that is used.

In still another example, the solvent-responsive bonding material 26B is applied to both the first side S₁ and the second side S₂ of the interposer 30, attaches the first side S₁ of the interposer 30 to the bonding region 40, and attaches the second side S₂ of the interposer 30 to the lid 22 or to the second substrate 24. Once the solvent-responsive bonding material 26B is applied and the components are in contact with one another, suitable curing and/or drying may be performed to achieve adhesion. In this example, it is to be understood that the material stack at the bonding regions 40, 40′ may be exposed to a single curing and/or drying exposure event to achieve adhesion. Alternatively, in this example, curing and/or drying exposure events may be performed after the solvent-responsive bonding material 26B is applied and the interposer 30 is in position, and again after the solvent-responsive bonding material 26B is applied and the lid 22 or the second substrate 24 is in position.

In one example, the heat-responsive bonding material 26C is applied to the bonding region 40 or to the first side S₁ of the interposer 30 and attaches the first side S₁ of the interposer 30 to the bonding region 40. In this example, the attaching of the lid 22 or the second substrate 24 to the second side S₂ of the interposer 30 involves applying a non-switchable bonding material 28 to the second side S₂; and contacting the lid 22 or the second substrate 24 with the non-switchable bonding material 28. Once the non-switchable bonding material 28 is applied and the components are in contact with one another, curing and/or drying may be performed depending upon the type of non-switchable bonding material 28 that is used.

In another example, the heat-responsive bonding material 26C is applied to the second side S₂ of the interposer 30 and attaches the second side S₂ of the interposer 30 to the lid 22 or to the second substrate 24. In this example, the attaching of the first side S₁ of the interposer 30 to the bonding region 40 involves applying the non-switchable bonding material 28 to the first side S₁ or to the bonding region 40 and then placing the first side S₁ and the bonding region 40 in contact with each other. Once the non-switchable bonding material 28 is applied and the components are in contact with one another, curing and/or drying may be performed depending upon the type of non-switchable bonding material 28 that is used.

In still another example, the heat-responsive bonding material 26C is applied to both the first side S₁ and the second side S₂ of the interposer 30, attaches the first side S₁ of the interposer 30 to the bonding region 40, and attaches the second side S₂ of the interposer 30 to the lid 22 or to the second substrate 24. Once the heat-responsive bonding material 26C is applied and the components are in contact with one another, suitable curing and/or drying may be performed to achieve adhesion. In this example, it is to be understood that the material stack at the bonding regions 40, 40′ may be exposed to a single curing and/or drying exposure event to achieve adhesion. Alternatively, in this example, curing and/or drying exposure events may be performed after the heat-responsive bonding material 26C is applied and the interposer 30 is in position, and again after the heat-responsive bonding material 26C is applied and the lid 22 or the second substrate 24 is in position. Curing and/or drying of the heat-responsive bonding material 26C takes place at a temperature that is below the temperature used to soften the microcapsules and gasify the liquid.

It is to be understood that the method(s) depicted in FIG. 3A through FIG. 3C may be performed using a substrate 14, 16 that includes any number of individual active region(s) 38, where the polymeric hydrogel 32 and the primers 42A, 42B are positioned within each active region 38 (e.g., in a lane 34, in depressions 36, or as functionalized pads 27). It is to be further understood that either the lid 22 or the second substrate 24 may be used with any of the substrates 14, 16.

### Methods of Using the Flow Cell

Various examples of a method of using the flow cell 10 will now be described in reference to FIG. 4A through FIG. 4D. Some of these examples generally involve exposing the light-switchable bonding material 26A to a predetermined wavelength of light L, thereby reducing adhesive properties of the light-switchable bonding material 26A; and separating the first substrate 14, 16 from the lid 22 or from the second substrate 24. Other of these examples generally involve exposing the solvent-responsive bonding material 26B to a predetermined solvent S for a predetermined time, thereby reducing adhesive properties of the solvent-responsive bonding material 26B, and separating the first substrate 14, 16 from the lid 22 or from the second substrate 24. Still other of these examples generally involve exposing the heat-responsive bonding material 26C to a predetermined temperature (represented by “H” in FIG. 4) for a predetermined time, thereby reducing adhesive properties of the heat-responsive bonding material 26C, and separating the first substrate 14, 16 from the lid 22 or from the second substrate 24.

The structure generated in FIG. 3C (e.g., a flow cell 10 including a lane 34 having a polymeric hydrogel 32 and primers 42A, 42B in the lane 34) is depicted in FIG. 4A. It is to be understood that while a particular example of the flow cell 10 is described in reference to FIG. 4A through FIG. 4D, any of the example flow cells 10 described herein may be used.

One example of the method of using the flow cell 10 proceeds from FIG. 4A to FIG. 4B. In this example, the light-switchable bonding material 26A is in contact with the first side S₁ of the interposer 30 and attaches the interposer 30 to the substrate 14, 16 at the first bonding region(s) 40. Further in this example, the non-switchable bonding material 28 is in contact with the second side S₂.

In this example, the predetermined wavelength of light L is introduced to the first bonding region(s) 40 from an excitation source that is positioned above the flow cell 10, below the flow cell 10, or at any desirable angle that will achieve the desired light L exposure. When the light L is introduced to the first bonding region(s) 40 from above the flow cell 10, it is to be understood that the lid 22 or the substrate 24, the non-switchable bonding material 28, and the interposer 30 are selected to be transparent to the light L. When the light L is introduced to the second bonding region(s) 40′ from below the flow cell 10, it is to be understood that the substrate 14, 16 is selected to be transparent to the light L. The predetermined wavelength of light L chemically modifies the light-switchable bonding material 26A at the first bonding region(s) 40, thereby reducing the adhesive properties of the light-switchable bonding material 26A. In some instances, the predetermined wavelength of light L is in the ultraviolet (UV) range. In other instances, the predetermined wavelength of light L is in the visible light range.

As shown in FIG. 4B, after the light-switchable bonding material 26A at the first bonding region(s) 40 has been chemically modified by the predetermined wavelength of light L, the interposer 30 (and the lid 22 or substrate 24 attached to the interposer 30) may be removed or detached from the first substrate 14, 16. In some instances, this removal process is facilitated by a separate cartridge/apparatus.

In other instances, the removal of the interposer 30 and the chemically modified light-switchable bonding material 26A is facilitated by a wedging process. During the wedging process, one or more wedge(s), such as a blade, is/are inserted between the interposer 30 and the substrate 14, 16 at the first bonding region(s) 40 and a force is applied to the wedge to aid in removal of the interposer 30 from the first bonding region(s) 40, thereby separating the first substrate 14, 16 from the lid 22 or from the second substrate 24 (where the interposer 30 remains attached to the lid 22 or to the second substrate 24). This wedging process may also remove the chemically modified light-switchable bonding material 26A from the first bonding region(s) 40. Alternatively, the chemically modified light-switchable bonding material 26A may be peeled from the first bonding region(s) 40 after wedging is performed.

In still other instances, the removal of the interposer 30 and the chemically modified light-switchable bonding material 26A is facilitated by a shearing process. During the shearing process, a lateral force is applied to the lid 22, to the second substrate 24, and/or to the interposer 30 to aid in removal of the interposer 30 from the first bonding region(s) 40, thereby separating the first substrate 14, 16 from the lid 22 or from the second substrate 24. As shown in the figure, during this process, the interposer 30 remains attached to the lid 22 or to the second substrate 24. This shearing process may also remove the chemically modified light-switchable bonding material 26A from the first bonding region(s) 40. Alternatively, the chemically modified light-switchable bonding material 26A may be peeled from the first bonding region(s) 40 after shearing is performed.

In yet further instances, the removal of the interposer 30 and the chemically modified light-switchable bonding material 26A involves passing a taut layer of metal foil through the interface between the first side S₁ of the interposer 30 and the bonding region(s) 40 of the substrate 14, 16. In these instances, the metal foil may have a width ranging from about 1 mm to about 50 mm. In a specific example, the aluminum foil has a width of about 25 mm. The metal foil may have a thickness ranging from about 1 μm to about 150 μm. In a specific example, the metal foil has a thickness ranging from about 15 μm to about 95 μm. The length of the metal foil may be adjusted to accommodate the length of the object being de-bonded (e.g., to accommodate the length of the components of the flow cell 10). The metal foil includes a corrosive or non-corrosive metal or metal-containing material. As one example, the metal foil may be stainless steel, e.g., 316 SS. The metal foil may have one or more serrated or non-serrated edges. The foil may have a micromachined knife blade angle ranging from about 10 degrees to about 15 degrees, or from about 15 degrees to about 20 degrees, or from about 20 degrees to about 25 degrees, or from about 25 degrees to about 30 degrees, or from about 35 degrees to about 40 degrees, or from about 40 degrees to about 45 degrees. Alternatively, the foil may have a symmetrically or asymmetrically pointed edge of any of these angles. The metal foil may include a coating thereon, where the coating includes titanium nitride or another suitable hardening material. In one example, the metal foil is coated with polytetrafluoroethylene or another suitable material that will reduce friction at surfaces of the metal foil.

The foil may be dispensed from a motorized roll, may be fixed in position, and may be tensioned using a retractable clamp that is positioned on either end of an object that is to be de-bonded, such as the flow cell 10. The metal foil may be incorporated into a cassette with a rotary drive mechanism that will enable lateral movement of an edge of a blade of the metal foil, e.g., towards a bonding region 40, to facilitate debonding. In some examples, after the metal foil is used to separate the substrate 14, 16 from the interposer 30, a small amount of residue of the chemically modified light-switchable bonding material 26A may remain on the substrate 14, 16 (e.g., at the bonding region(s) 40). In these examples, a solvent and/or physical polishing process may be used to remove the residual adhesive material 26A from the bonding region(s) 40. It is to be understood that the foil may be capable of removing light-switchable bonding materials 26A from bonding regions 40, even when the light-switchable bonding materials 26A are not exposed to light L from an external source.

Another example of a method of using the flow cell 10 still proceeds from FIG. 4A to FIG. 4B, but in this example, the solvent-responsive bonding material 26B is utilized. In this example, the solvent-responsive bonding material 26B is in contact with the first side S₁ of the interposer 30 and attaches the interposer 30 to the substrate 14, 16 at the first bonding region(s) 40.

In this example, the predetermined solvent S is introduced to the first bonding region(s) 40, such that the solvent-responsive bonding material 26B is exposed to the solvent S for a predetermined time. The length of the predetermined time will depend, in part, upon the material used for the solvent-responsive bonding material 26B and the solvent S that is used. In some examples, the solvent-responsive bonding material 26B may be exposed to the solvent S for about 7 days at ambient temperature. The predetermined solvent S chemically modifies the solvent-responsive bonding material 26B at the first bonding region(s) 40 and/or causes the solvent-responsive bonding material 26B to swell, thereby reducing the adhesive properties of the solvent-responsive bonding material 26B or resulting in a physical change within the solvent-responsive bonding material 26B (e.g., expansion) that separates the interposer 30 from the substrate 14, 16. The predetermined solvent S that is selected to modify the solvent-responsive bonding material 26B will depend upon the material used for the solvent-responsive bonding material 26B. In examples, the solvent S may be isopropyl alcohol, an organic solvent, a buffer, or any other suitable solvent S that will modify the solvent-responsive bonding material 26B.

The introducing of the predetermined solvent S may be performed by introducing the solvent S into the flow channel(s) 12 and allowing it to incubate for the predetermined time. The flow cell 10 itself may also be introduced to a solvent bath that will expose the outermost bonding regions 40, 40′ (e.g., at the perimeter of the flow cell 10) to the solvent S.

As shown in FIG. 4B, after the solvent-responsive bonding material 26B at the first bonding region(s) 40 has been chemically modified by the predetermined solvent S, the interposer 30 (and the lid 22 or substrate 24 attached to the interposer 30) is removed from the first substrate 14, 16. In some instances, this removal process is facilitated by a separate cartridge/apparatus.

In other instances, the removal of the interposer 30 and the chemically modified solvent-responsive bonding material 26B is facilitated by a wedging process or a shearing process, either of which is performed in a similar manner as described in reference to the light-switchable bonding material 26A as described in reference to FIG. 4B.

In yet further instances, the removal of the interposer 30 and the chemically modified solvent-responsive bonding material 26B involves passing a taut layer of metal foil through the interface between the first side S₁ of the interposer 30 and the bonding region(s) 40 of the substrate 14, 16. In these instances, the metal foil may have a width ranging from about 1 μm to about 50 μm. In a specific example, the metal foil has a width of about 25 μm. The length of the metal foil may be adjusted to accommodate the length of the object being de-bonded (e.g., to accommodate the length of the components of the flow cell 10). The metal foil may be stainless steel, e.g., 316 SS. The foil may be dispensed from a motorized roll, and the foil may be tensioned using a retractable clamp that is positioned on either end of an object that is to be de-bonded. In some examples, after the metal foil is used to separate the substrate 14, 16 from the interposer 30, a small amount of residue of the chemically modified solvent-responsive bonding material 26B may be left behind on the substrate 14, 16 (e.g., at the bonding region(s) 40). In these examples, a solvent and/or physical polishing process may be used to remove the residual adhesive material 26B from the bonding region(s) 40. It is to be understood that the foil may be capable of removing solvent-responsive bonding materials 26B from bonding regions 40, even when the solvent-responsive bonding materials 26B are not exposed to a solvent S.

Another example of a method of using the flow cell 10 still proceeds from FIG. 4A to FIG. 4B, but in this example, the heat-responsive bonding material 26C is utilized. In this example, the heat-responsive bonding material 26C is in contact with the first side S₁ of the interposer 30 and attaches the interposer 30 to the substrate 14, 16 at the first bonding region(s) 40.

In this example, a predetermined amount of heat “H” (quantified by a temperature or a range of temperatures) is introduced to the first bonding region(s) 40, such that the heat-responsive bonding material 26C is exposed to the temperature/heat H for a predetermined time. The length of the predetermined time will depend, in part, upon the material used for the heat-responsive bonding material 26C and the temperature of the heat H that is introduced thereto. The predetermined amount of heat H chemically modifies the heat-responsive bonding material 26C at the first bonding region(s) 40, thereby reducing the adhesive properties of the heat-responsive bonding material 26C. When the heat-responsive bonding material 26C includes microspheres, as described herein, the heat H is capable of initiating softening of the outer shell and gasification of the liquid within the microspheres, which increases the volume of the heat-responsive bonding material 26C and reduces adhesive properties of the heat-responsive bonding material 26C.

The introduction of the predetermined amount of heat H may involve placing the flow cell 10 on a heated surface (such as a hot plate) and/or may involve exposing the flow cell 10 to heat from an external thermal radiation source. As an example, the external thermal radiation source may be a laser (e.g., pulsed or continuous-wave) that delivers wavelengths of light of about 308 nm, or about 355 nm, or about 1064 nm. In an example in which the laser delivers wavelengths of light of about 1064 nm, the laser may have a diameter of about 0.4 mm and may deliver 20 watts of power.

As shown in FIG. 4B, after the heat-responsive bonding material 26C at the first bonding region(s) 40 has been chemically modified by the predetermined amount of heat H, the interposer 30 (and the lid 22 or substrate 24 attached to the interposer 30) is removed from the first substrate 14, 16. In some instances, this removal process is facilitated by a separate cartridge/apparatus.

In other instances, the removal of the interposer 30 and the chemically modified heat-responsive bonding material 26C is facilitated by a wedging process or a shearing process, either of which is performed in a similar manner as described in reference to the light-switchable bonding material 26A or the solvent-responsive bonding material 26B as described in reference to FIG. 4B.

In yet further instances, the removal of the interposer 30 and the chemically modified heat-responsive bonding material 26C involves passing a taut layer of metal foil through the interface between the first side S₁ of the interposer 30 and the bonding region(s) 40 of the substrate 14, 16. In these instances, the metal foil may have a width ranging from about 1 μm to about 50 μm. In a specific example, the metal foil has a width of about 25 μm. The length of the metal foil may be adjusted to accommodate the length of the object being de-bonded (e.g., to accommodate the length of the components of the flow cell 10). The metal foil may be stainless steel, e.g., 316 SS. The foil may be dispensed from a motorized roll, and the foil may be tensioned using a retractable clamp that is positioned on either end of an object that is to be de-bonded. In some examples, after the metal foil is used to separate the substrate 14, 16 from the interposer 30, a small amount of residue of the chemically modified heat-responsive bonding material 26C may be left behind on the substrate 14, 16 (e.g., at the bonding region(s) 40). In these examples, a solvent and/or physical polishing process may be used to remove the residual adhesive material 26C from the bonding region(s) 40. It is to be understood that the foil may be capable of removing heat-responsive bonding materials 26C from the bonding regions 40, even when the heat responsive-bonding materials 26C have not been exposed to heat H from an external source.

Another example of the method of using the flow cell 10 proceeds from FIG. 4A to FIG. 4C. In this example, the light-switchable bonding material 26A is in contact with the second side S₂ of the interposer 30 and attaches the interposer 30 to the lid 22 or to the second substrate 24 at the second bonding region(s) 40′. Further in this example, the non-switchable bonding material 28 is in contact with the first side S₁.

In this example, the predetermined wavelength of light L is introduced to the second bonding region(s) 40′ from an excitation source that is positioned above the flow cell 10, below the flow cell 10, or at any desirable angle that will achieve the desired light L exposure. When the light L is introduced to the second bonding region(s) 40′ from above the flow cell 10, it is to be understood that the lid 22 or the substrate 24 is selected to be transparent to the light L. When the light L is introduced to the second bonding region(s) 40′ from below the flow cell 10, it is to be understood that the substrate 14, 16, the non-switchable bonding material 28, and the interposer 30 are selected to be transparent to the light L. The predetermined wavelength of light L chemically modifies the light-switchable bonding material 26A at the second bonding region(s) 40′, thereby reducing the adhesive properties of the light-switchable bonding material 26A. In some instances, the predetermined wavelength of light L is in the ultraviolet (UV) range. In other instances, the predetermined wavelength of light L is in the visible light range.

As shown in FIG. 4C, after the light-switchable bonding material 26A at the second bonding region(s) 40′ has been chemically modified by the predetermined wavelength of light L, the lid 22 or substrate 24 may be removed/detached from the first substrate 14, 16 and from the interposer 30. As further shown in FIG. 4C, the removal of the lid 22 or the substrate 24 from the interposer 30 leaves the interposer 30 attached to the first substrate 14, 16. In some instances, this removal process is facilitated by a separate cartridge/apparatus.

In other instances, the removal of the lid 22 or the second substrate 24 and the chemically modified light-switchable bonding material 26A is facilitated by a wedging process. During the wedging process, one or more wedge(s) is/are inserted between the interposer 30 and the lid 22 or the second substrate 24 at the second bonding region(s) 40′ and a force is applied to the wedge to aid in removal of the interposer 30, thereby separating the first substrate 14, 16 (having the interposer 30 attached thereto) from the lid 22 or from the second substrate 24. This wedging process may also remove the chemically modified light-switchable bonding material 26A from the second bonding region(s) 40′. Alternatively, the chemically modified light-switchable bonding material 26A may be peeled from the second bonding region(s) 40′ after wedging is performed.

In still other instances, the removal of the lid 22 or the second substrate 24 and the removal of the chemically modified light-switchable bonding material 26A is facilitated by a shearing process. During the shearing process, a lateral force is applied to the lid 22 or to the second substrate 24 to aid in removal of the lid 22 or the second substrate 24 from the second bonding region(s) 40′, thereby separating the second substrate 24 or the lid 22 from the interposer 30. As shown in the figure, during this process, the interposer 30 remains attached to the first substrate 14, 16. This shearing process may also remove the chemically modified light-switchable bonding material 26A from the second bonding region(s) 40′. Alternatively, the chemically modified light-switchable bonding material 26A may be peeled from the second bonding region(s) 40′ after shearing is performed.

In yet further instances, the removal of the lid 22 or the second substrate 24 and the removal of the chemically modified light-switchable bonding material 26A involves passing a taut layer of metal foil through the interface between the second side S₂ of the interposer 30 and the lid 22 or second substrate 24 (e.g., at the bonding region(s) 40′). In these instances, the metal foil may have a width ranging from about 1 μm to about 50 μm. In a specific example, the metal foil has a width of about 25 μm. The length of the metal foil may be adjusted to accommodate the length of the object being de-bonded (e.g., to accommodate the length of the components of the flow cell 10). The foil may be stainless steel, e.g., 316 SS. The foil may be dispensed from a motorized roll, and the foil may be tensioned using a retractable clamp that is positioned on either end of an object that is to be de-bonded. In some examples, after the metal foil is used to separate the lid 22 or second substrate 24 from the interposer 30, a small amount of residue of the chemically modified light-switchable bonding material 26A may be left behind on the second side S₂ of the interposer 30. In these examples, a solvent and/or physical polishing process may be used to remove the residual adhesive material 26A from the bonding region(s) 40′. The foil may be capable of removing the light-switchable bonding material 26A from the bonding region(s) 40′, even when the light-switchable bonding material 26A has not been exposed to light L from an external source.

Another example of the method of using the flow cell still proceeds from FIG. 4A to FIG. 4C, but in this example, the solvent-responsive bonding material 26B is used. In this example, the solvent-responsive bonding material 26B is in contact with the second side S₂ of the interposer 30 and attaches the interposer 30 to the lid 22 or to the second substrate 24 at the second bonding region(s) 40′. Further in this example, the non-switchable bonding material 28 is in contact with the first side S₁.

In this example, the predetermined solvent S is introduced to the bonding region(s) 40′, such that the solvent-responsive bonding material 26B is exposed to the solvent S for a predetermined time. The length of the predetermined time will depend, in part, upon the material used for the solvent-responsive bonding material 26B and the solvent S that is used. The predetermined solvent S chemically modifies the solvent-responsive bonding material 26B at the second bonding region(s) 40′, thereby reducing the adhesive properties of the solvent-responsive bonding material 26B. The predetermined solvent S that is used to modify the solvent-responsive bonding material 26B will depend upon the material used for the solvent-responsive bonding material 26B. The solvent S may be any suitable example disclosed herein.

As shown in FIG. 4C, after the solvent-responsive bonding material 26B at the second bonding region(s) 40′ has been chemically modified by the predetermined solvent S, the lid 22 or substrate 24 attached to the interposer 30 is removed from the first substrate 14, 16 and from the interposer 30. As further shown in FIG. 4C, the removal of the lid 22 or the substrate 24 from the interposer 30 leaves the interposer 30 attached to the first substrate 14, 16 (e.g., at the first bonding region(s) 40).

In some instances, this removal process is facilitated by a separate cartridge/apparatus. In other instances, the removal of the interposer 30 and the chemically modified solvent-responsive bonding material 26B is facilitated by a wedging process or a shearing process, either of which is performed in a similar manner as described in reference to the light-switchable bonding material 26A as described in reference to FIG. 4C.

In yet further instances, the removal of the lid 22 or the second substrate 24 and the removal of the chemically modified solvent-responsive bonding material 26B involves passing a taut layer of metal foil through the interface between the second side S₂ of the interposer 30 and the lid 22 or second substrate 24 (e.g., at the bonding region(s) 40′). In these instances, the metal foil may have a width ranging from about 1 μm to about 50 μm. In a specific example, the metal foil has a width of about 25 μm. The length of the metal foil may be adjusted to accommodate the length of the object being de-bonded (e.g., to accommodate the length of the components of the flow cell 10). The metal foil may include stainless steel, e.g., 316 SS. The foil may be dispensed from a motorized roll, and the foil may be tensioned using a retractable clamp that is positioned on either end of an object that is to be de-bonded. In some examples, after the metal foil is used to separate the lid 22 or second substrate 24 from the interposer 30, a small amount of residue of the chemically modified solvent-responsive bonding material 26B may be left behind on the second side S₂ of the interposer 30. In these examples, a solvent and/or physical polishing process may be used to remove the residual adhesive material 26B from the bonding region(s) 40′. The foil may be capable of removing the solvent-responsive bonding material 26B from the bonding region(s) 40′, even when the solvent-responsive bonding material 26B has not been exposed to the solvent S.

Another example of the method of using the flow cell 10 still proceeds from FIG. 4A to FIG. 4C, but in this example, the heat-responsive bonding material 26C is used. In this example, the heat-responsive bonding material 26C is in contact with the second side S₂ of the interposer 30 and attaches the interposer 30 to the lid 22 or to the second substrate 24 at the second bonding region(s) 40′. Further in this example, the non-switchable bonding material 28 is in contact with the first side S₁.

In this example, the predetermined amount of heat H is introduced to the bonding region(s) 40′, such that the heat-responsive bonding material 26C is exposed to the heat H for a predetermined time. The length of the predetermined time will depend, in part, upon the material used for the heat-responsive bonding material 26C and the temperature of the heat H that is used. The predetermined amount of heat H chemically modifies the heat-responsive bonding material 26C at the second bonding region(s) 40′, thereby reducing the adhesive properties of the heat-responsive bonding material 26C. The predetermined amount of heat H that is used to modify the heat-responsive bonding material 26C will depend, in part, upon the material used for the heat-responsive bonding material 26C.

As shown in FIG. 4C, after the heat-responsive bonding material 26C at the second bonding region(s) 40′ has been chemically modified by the predetermined amount of heat H, the lid 22 or substrate 24 attached to the interposer 30 is removed from the first substrate 14, 16 and from the interposer 30. As further shown in FIG. 4C, the removal of the lid 22 or the substrate 24 from the interposer 30 leaves the interposer 30 attached to the first substrate 14, 16 (e.g., at the first bonding region(s) 40).

In some instances, this removal process is facilitated by a separate cartridge/apparatus. In other instances, the removal of the interposer 30 and the chemically modified heat-responsive bonding material 26C is facilitated by a wedging process or a shearing process, either of which is performed in a similar manner as described in reference to the light-switchable bonding material 26A or the solvent-responsive bonding material 26B as described in reference to FIG. 4C.

In yet further instances, the removal of the lid 22 or the second substrate 24 and the removal of the heat-responsive bonding material 26C involves passing a taut layer of metal foil through the interface between the second side S₂ of the interposer 30 and the lid 22 or second substrate 24 (e.g., at the bonding region(s) 40′). In these instances, the metal foil may have a width ranging from about 1 μm to about 50 μm. In a specific example, the metal foil has a width of about 25 μm. The length of the metal foil may be adjusted to accommodate the length of the object being de-bonded (e.g., to accommodate the length of the components of the flow cell 10). The metal foil may include stainless steel, e.g., 316 SS. The foil may be dispensed from a motorized roll, and the foil may be tensioned using a retractable clamp that is positioned on either end of an object that is to be de-bonded. In some examples, after the metal foil is used to separate the lid 22 or second substrate 24 from the interposer 30, a small amount of residue of the chemically modified heat-responsive bonding material 26C may be left behind on the second side S₂ of the interposer 30. In these examples, a solvent and/or physical polishing process may be used to remove the residual adhesive material 26C from the bonding region(s) 40′. The foil may be capable of removing the heat-responsive bonding material 26C from the bonding region(s) 40′, even when the heat-responsive bonding material 26C has not been exposed to light L from an external source.

The thin layer of metal foil may also be used to separate flow cell components from one another that are bonded using the non-switchable bonding material 28.

As such, regardless of whether the light-switchable bonding material 26A the solvent-responsive bonding material 26B, the heat-responsive bonding material 26C, or the non-switchable bonding material 28 is/are used in the flow cell 10, the separating of the first substrate 14, 16 from the lid 22 or from the second substrate 24 may involve passing a taut layer of metal having a width ranging from about 1 mm to about 50 mm through: the interface between the first side S₁ and the bonding region(s) 40, or the interface between the second side S₂ and the lid 22 or the second substrate 24.

Another example of the method of using the flow cell 10 proceeds from FIG. 4A to FIG. 4D. In this example, the light-switchable bonding material 26A is in contact with both the first side S₁ and the second side S₂ of the interposer 30, attaches the first side S₁ of the interposer 30 to the first bonding region(s) 40, and attaches the second side S₂ of the interposer 30 to the lid 22 or to the second substrate 24 (e.g., at the second bonding region(s) 40′).

In this example, the predetermined wavelength of light L is introduced to the first and second bonding region(s) 40, 40′ from an excitation source that is positioned above the flow cell 10, below the flow cell 10, or at any desirable angle that will achieve the desired light L exposure. When the light L is introduced to the first and second bonding region(s) 40, 40′ from above the flow cell 10, it is to be understood that the lid 22 or the substrate 24 and the interposer 30 is selected to be transparent to the light L. When the light L is introduced to the first and second bonding region(s) 40, 40′ from below the flow cell 10, it is to be understood that the substrate 14, 16 and the interposer 30 is selected to be transparent to the light L. The predetermined wavelength of light L chemically modifies the light-switchable bonding material 26A at the first and second bonding region(s) 40, 40′, thereby reducing the adhesive properties of the light-switchable bonding material 26A. In some instances, the predetermined wavelength of light L is in the ultraviolet (UV) range. In other instances, the predetermined wavelength of light L is in the visible light range.

As shown in FIG. 4D, after the light-switchable bonding material 26A at the first and second bonding region(s) 40, 40′ has been chemically modified by the predetermined wavelength of light L, the interposer 30 may be removed from the first substrate 14, 16 (at the first bonding region(s) 40), and the lid 22 or the second substrate 24 may be removed from the interposer 30 (at the second bonding region(s) 40′). In some instances, this removal process is facilitated by a separate cartridge/apparatus.

In other instances, the removal of the interposer 30, the lid 22 or the second substrate 24, and the chemically modified light-switchable bonding material 26A is facilitated by a wedging process. During the wedging process, one or more wedge(s) are inserted between the interposer 30 and the substrate 14, 16 and are inserted between the interposer 30 and the lid 22 or the substrate 24 at the respective bonding region(s) 40, 40′. A force is then applied to the wedge(s) to aid in removal of either the lid 22 or substrate 24 or the substrate 14, 16 from the interposer 30. This wedging process may also remove the chemically modified light-switchable bonding material 26A from each of the first and second bonding region(s) 40, 40′. Alternatively, the chemically modified light-switchable bonding material 26A may be peeled from each of the first and second bonding region(s) 40, 40′ after wedging is performed.

In still other instances, the removal of the interposer 30, the lid 22 or the second substrate 24, and the chemically modified light-switchable bonding material 26A is facilitated by a shearing process. During the shearing process, a lateral force is applied to the lid 22, to the second substrate 24, and/or to the interposer 30 to aid in removal of the interposer 30 from the first bonding region(s) 40, thereby separating the first substrate 14, 16 from the lid 22 or from the second substrate 24. As shown in the figure, during this process, the interposer 30 detaches from both the first substrate 14, 16 and from the lid 22 or second substrate 24. This shearing process may also remove the chemically modified light-switchable bonding material 26A from the first and second bonding region(s) 40, 40′. Alternatively, the chemically modified light-switchable bonding material 26A may be peeled from each of the first and second bonding region(s) 40, 40′ after shearing is performed.

It is to be understood that the removal of the interposer 30, the lid 22, the second substrate 24, and the chemically modified light-switchable bonding material 26A may also be facilitated by passing a taut layer of metal foil through the respective interfaces between the (i) the first side S₁ of the interposer 30 and the bonding region(s) 40 of the substrate 14, 16, or between (ii) the second side S₂ of the interposer 30 and the lid 22 or the second substrate 24 (at the second bonding region(s) 40′). As described, the foil may be capable of removing the light-switchable bonding material 26A from the bonding region(s) 40, 40′, even when the light-switchable bonding material 26A has not been exposed to light L from an external source.

Another example of the method of using the flow cell 10 still proceeds from FIG. 4A to FIG. 4D, but in this example, the solvent-responsive bonding material 26B is used. In this example, the solvent-responsive bonding material 26B is in contact with both the first side S₁ and the second side S₂ of the interposer 30, attaches the first side S₁ of the interposer 30 to the first substrate 14, 16 (e.g., at the first bonding region(s) 40), and attaches the second side S₂ of the interposer 30 to the lid 22 or to the second substrate 24 (e.g., at the second bonding region(s) 40′).

In this example, the predetermined solvent S is introduced to the first and second bonding region(s) 40, 40′, such that the solvent-responsive bonding material 26B is exposed to the solvent S for a predetermined time. The length of the predetermined time will depend, in part, upon the material used for the solvent-responsive bonding material 26B and the solvent S that is used. The predetermined solvent S chemically modifies the solvent-responsive bonding material 26B at the first and second bonding region(s) 40,40′, thereby reducing the adhesive properties of the solvent-responsive bonding material 26B. The predetermined solvent S that is used to modify the solvent-responsive bonding material 26B will depend upon the material used for the solvent-responsive bonding material 26B. The solvent S may be any suitable example disclosed herein.

As shown in FIG. 4D, after the solvent-responsive bonding material 26B at the first and second bonding region(s) 40, 40′ has been chemically modified by the predetermined solvent S, the interposer 30 may be detached from the first substrate 14, 16 (e.g., at the first bonding region(s) 40), and the interposer 30 may also be detached from the lid 22 or from the second substrate 24 (e.g., at the second bonding region(s) 40′). In some instances, this removal process is facilitated by a separate cartridge/apparatus. In other instances, the removal of the interposer 30 and the chemically modified solvent-responsive bonding material 26B is facilitated by a wedging process or a shearing process, either of which is performed in a similar manner as described in reference to the light-switchable bonding material 26A as described in reference to FIG. 4D.

It is to be understood that the removal of the interposer 30, the lid 22, the second substrate 24, and the chemically modified solvent-responsive bonding material 26B may also be facilitated by passing a taut layer of metal foil through the respective interfaces between the (i) the first side S₁ of the interposer 30 and the bonding region(s) 40 of the substrate 14, 16, or between (ii) the second side S₂ of the interposer 30 and the lid 22 or the second substrate 24 (at the second bonding region(s) 40′).

Another example of the method of using the flow cell 10 still proceeds from FIG. 4A to FIG. 4D, but in this example, the heat-responsive bonding material 26C is used. In this example, the heat-responsive bonding material 26C is in contact with both the first side S₁ and the second side S₂ of the interposer 30, attaches the first side S₁ of the interposer 30 to the first substrate 14, 16 (e.g., at the first bonding region(s) 40), and attaches the second side S₂ of the interposer 30 to the lid 22 or to the second substrate 24 (e.g., at the second bonding region(s) 40′).

In this example, the predetermined amount of heat H is introduced to the first and second bonding region(s) 40, 40′, such that the heat-responsive bonding material 26C is exposed to the heat H for a predetermined amount of time. The length of the predetermined time will depend, in part, upon the material used for the heat-responsive bonding material 26C and the temperature of the heat H that is used. The predetermined amount of heat H chemically modifies the heat-responsive bonding material 26C at the first and second bonding region(s) 40,40′, thereby reducing the adhesive properties of the heat-responsive bonding material 26C. The predetermined amount of heat that is used to modify the heat-responsive bonding material 26C will depend upon the material used for the heat-responsive bonding material 26C.

As shown in FIG. 4D, after the heat-responsive bonding material 26C at the first and second bonding region(s) 40, 40′ has been chemically modified by the predetermined amount of heat H, the interposer 30 may be detached from the first substrate 14, 16 (e.g., at the first bonding region(s) 40), and the interposer 30 may also be detached from the lid 22 or from the second substrate 24 (e.g., at the second bonding region(s) 40′). In some instances, this removal process is facilitated by a separate cartridge/apparatus. In other instances, the removal of the interposer 30 and the chemically modified heat-responsive bonding material 26C is facilitated by a wedging process or a shearing process, either of which is performed in a similar manner as described in reference to the light-switchable bonding material 26A or the solvent-responsive bonding material 26B as described in reference to FIG. 4D.

It is to be understood that the removal of the interposer 30, the lid 22, the second substrate 24, and the chemically modified heat-responsive bonding material 26C may also be facilitated by passing a taut layer of metal foil through the respective interfaces between the (i) the first side S₁ of the interposer 30 and the bonding region(s) 40 of the substrate 14, 16, or between (ii) the second side S₂ of the interposer 30 and the lid 22 or the second substrate 24 (at the second bonding region(s) 40′), similar to the manner as described in reference to the light-switchable bonding material 26A or the solvent-responsive bonding material 26B in FIG. 4D. As described, the foil may be capable of removing the heat-responsive bonding material 26C from the bonding region(s) 40, 40′, even when the heat-responsive bonding material 26C has not been exposed to heat H from an external source.

Accordingly, some examples of the method of using the flow cell 10 involve separating the first substrate 14, 16 from the lid 22 or from the second substrate 24, wherein the light-switchable bonding material 26A is in contact with the first side S₁ of the interposer 30 and the separating is performed at the first bonding region(s) 40 (see FIG. 4B). Some other examples of the method of using the flow cell 10 involve separating the first substrate 14, 16 from the lid 22 or from the second substrate 24, wherein the solvent-responsive bonding material 26B is in contact with the first side S₁ of the interposer 30 and the separating is performed at the first bonding region(s) 40 (see FIG. 4B). Further examples of the method of using the flow cell 10 involve separating the first substrate 14, 16 from the lid 22 or from the second substrate 24, wherein the heat-responsive bonding material 26C is in contact with the first side S₁ of the interposer 30 and the separating is performed at the first bonding region(s) 40 (see FIG. 4B).

Further, some examples of the method of using the flow cell 10 involve separating the first substrate 14, 16 from the lid 22 or from the second substrate 24, wherein the light-switchable bonding material 26A is in contact with the second side S₁ of the interposer 30 and the separating is performed at the second bonding region(s) 40′ (see FIG. 4C). Some other examples of the method of using the flow cell 10 involve separating the first substrate 14, 16 from the lid 22 or from the second substrate 24, wherein the solvent-responsive bonding material 26B is in contact with the second side S₂ of the interposer 30 and the separating is performed at the second bonding region(s) 40′ (see FIG. 4C). Still other examples of the method of using the flow cell 10 involve separating the first substrate 14, 16 from the lid 22 or from the second substrate 24, wherein the heat-responsive bonding material 26C is in contact with the second side S₂ of the interposer 30 and the separating is performed at the second bonding region(s) 40′ (see FIG. 4C).

Still further, some examples of the method of using the flow cell 10 involve separating the first substrate 14, 16 from the lid 22 or from the second substrate 24, wherein the light-switchable bonding material 26A is in contact with the first side S₁ and the second side S₂ of the interposer 30, and the separating is performed at the first and second bonding region(s) 40, 40′ (see FIG. 4D). Some other examples of the method of using the flow cell 10 involve separating the first substrate 14, 16 from the lid 22 or from the second substrate 24, wherein the solvent-responsive bonding material 26B is in contact with the first side S₁ and the second side S₂ of the interposer 30, and the separating is performed at the first and second bonding region(s) 40, 40′ (see FIG. 4D). Still other examples of the method of using the flow cell 10 involve separating the first substrate 14, 16 from the lid 22 or from the second substrate 24, wherein the heat-responsive bonding material 26C is in contact with the first side S₁ and the second side S₂ of the interposer 30, and the separating is performed at the first and second bonding region(s) 40, 40′ (see FIG. 4D).

While FIG. 4A through FIG. 4D collectively describe examples in which the chemically modified light-switchable bonding material 26A, the chemically modified solvent-responsive bonding material 26B, or the chemically modified heat-responsive bonding material 26C is involved in the separating of the components of the flow cell 10, it is to be understood that at least some of the techniques described herein are applicable to the non-switchable bonding material 28 as well. For example, as described herein, components of the flow cell 10 that are bonded together using the non-switchable bonding material 28 (e.g., the interposer 30, the substrate 14, 16, the lid 22, and/or the second substrate 24) may be separated from one another using the taut layer of metal foil described herein. As such, one example of a method of using a flow cell involves passing a taut layer of metal having a width ranging from about 1 μm to about 50 μm through an adhesive material that is positioned between (a) bonding region(s) of a first substrate 14, 16 and a first side S₁ of an interposer 30, or through an adhesive material that is positioned between a second side S₂ of the interposer 30 and a lid 22 or a second substrate 24, thereby: separating the first side S₁ of the interposer 30 from the first substrate 14, 16, or separating the lid 22 or the second substrate 24 from the second side S₂ of the interposer 30. In this example, the adhesive material may be the non-switchable bonding material 28, and the metal foil may be stainless steel (e.g., 316 SS).

The light-switchable bonding material(s) 26A, the solvent-responsive bonding material(s) 26B, the heat-responsive bonding material(s) 26C, and the non-switchable bonding material 28 disclosed herein may also be included in a biological sequencing kit, an example of which will now be described.

### Biological Sequencing Kit

An example of a biological sequencing kit contains a flow cell 10 including: a first substrate 14, 16 having an active region 38 and a bonding region 40 that is spatially separate from the active region 38; a polymeric hydrogel 32 positioned at the active region 38; an interposer 30 having a first side S₁ that is attached to the bonding region 40 and having a second side S₂ that is opposed to the first side S₁; a light-switchable bonding material 26A in contact with at least one of the first side S₁ or the second side S₂, the light-switchable bonding material 26A exhibiting a decrease in adhesive properties upon exposure to visible light; and a lid 22 or a second substrate 24 attached to the second side S₂ of the interposer 30; and a visible light blocking storage container housing the flow cell 10.

The flow cell 10 in the kit may include any patterned or unpatterned structure disclosed herein. For example, the flow cell 10 may include a lane 34 having surface chemistry therein (similar to FIG. 11B), or the flow cell 10 may include functionalized pads 27 (similar to FIG. 1C), or the flow cell 10 may include depressions 36 having surface chemistry therein (similar to FIG. 2A through FIG. 2D). In this example, the light-switchable bonding material 26A exhibits a decrease in adhesive properties upon exposure to visible light. As such, the flow cell 10 is stored in a visible light blocking storage container. The visible light blocking storage container may be made of, for example, black plastic, amber glass, amber plastic, tin, or aluminum.

Another example of the biological sequencing kit contains a flow cell 10 including: a first substrate 14, 16 having an active region 38 and a bonding region 40 that is spatially separate from the active region 38; a polymeric hydrogel 32 positioned at the active region 38; an interposer 30 having a first side S₁ that is attached to the bonding region 40 and having a second side S₂ that is opposed to the first side S₁; a solvent-responsive bonding material 26B on at least one of the first side S₁ or the second side S₂; and a lid 22 or a second substrate 24 attached to the second side S₂ of the interposer 30; and a solvent S to which the solvent-responsive bonding material 26B is sensitive. The solvent S, when included in the kit, may be an aqueous solvent, or an organic solvent, and its selection will depend in part upon the solvent-responsive bonding material 26B included in the flow cell 10 in the kit.

Another example of the biological sequencing kit contains a flow cell 10 including: a first substrate 14, 16 having an active region 38 and a bonding region 40 that is spatially separate from the active region 38; a polymeric hydrogel 32 positioned at the active region 38; an interposer 30 having a first side S₁ that is attached to the bonding region 40 and having a second side S₂ that is opposed to the first side S₁; a heat-responsive bonding material 26C on at least one of the first side S₁ or the second side S₂; and a lid 22 or a second substrate 24 attached to the second side S₂ of the interposer 30; and a heat source to provide a predetermined amount of heat H to which the heat-responsive bonding material 26C is sensitive. The heat source, when included in the kit, may be a thermal radiation source, such as an infrared-light source.

Any example of the kit may further include reagents for sequencing operations, such as incorporation mixes, wash solutions, buffers, catalysts, liquid carriers, and the like.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

A condition screening was performed using 12 strips of adhesive applied to three individual glass substrates (i.e., Corning® EAGLE XG® glass substrates, CEXG). One of the CEXG substrates did not include any additional surface modifications (Trials 1-4). Another of the CEXG substrates was imprinted using nanoimprint lithography and was then exposed to ashing and silane activation, followed by deposition of PAZAM (Trials 5-8). Another of the CEXG substrates was imprinted using nanopimrint lithography and was then exposed to ashing and silane activation alone (Trials 9-12).

Each of the adhesives applied to the three substrates included a light-switchable bonding material (i.e., a UV-curable polymer and a photoinitiator). The light-switchable bonding material that was used exhibits increased properties of adhesion upon exposure to visible light and decreased properties of adhesion upon exposure to UV light. Thus, visible light was used to cure the adhesive and form the substrates.

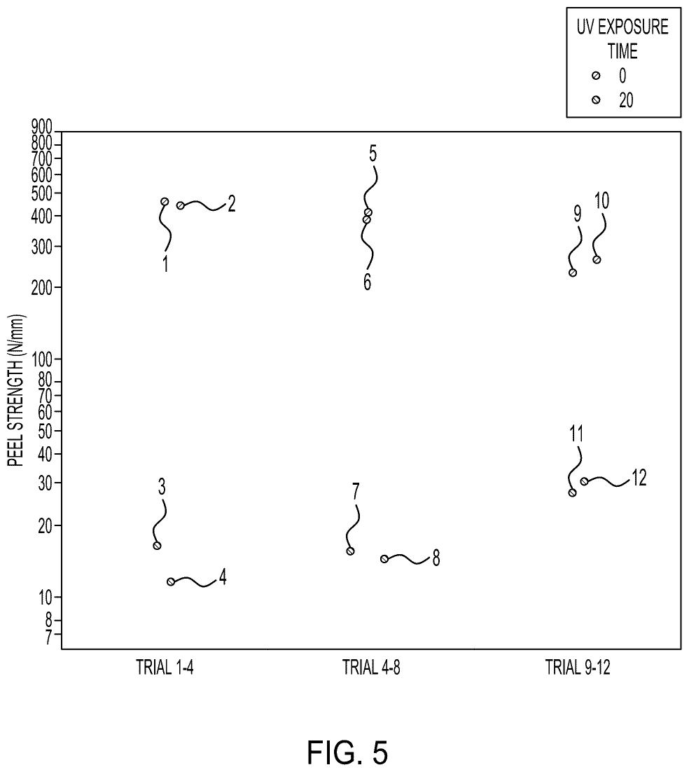

An initial peel strength measurement (in newtons/meter) was taken for trials 1, 2, 5, 6, 9, and 10 to determine the force required to separate the adhesive from the substrate. The results of these initial peel strength measurements for these substrates are shown in FIG. 5. As can be seen in FIG. 5, without any UV exposure, the respective adhesives for each of trials 1, 2, 5, 6, 9, and 10 exhibited a high peel strength (e.g., 200 N/m or greater).

Each of the substrates was exposed to UV light for 20 seconds. A peel strength measurement was then taken, which produced the results for trials 3, 4, 7, 8, 11, and 12. These peel strength measurements are also shown in FIG. 5. As can be seen in the figure, after UV exposure, the adhesives in trials 3, 4, 7, 8, 11, and 12 exhibited a significantly lower peel strength (about 90% lower) than the peel strength that was observed for trials 1, 2, 5, 6, 9, and 10, during which the adhesives had not been not exposed to UV light.

These results demonstrate that the light-switchable bonding materials disclosed herein may be used to detach flow cell components, such as interposers and substrates, from one another by exposing the light-switchable bonding materials to predetermined light wavelengths. After exposure to the predetermined light wavelengths, the peel strength of the interposers can be significantly decreased (by orders of magnitude), such that far less force is required to remove the interposers (from their respective substrates) than the amount of force that was required without exposure to the light wavelengths.

### Example 2

Eight example flow cells (Flow cells 1-2 through 8-2) were prepared. Each of the substrates of the flow cells was a planar multi-layer substrate having a resin over a glass base support. The resin layer was exposed to silanization and plasma ashing. An adhesive was applied to the planar surfaces of different substrates, and different interposers were positioned on the adhesives. A lid was then positioned on each of the interposers, using a solvent-responsive bonding material positioned at a bonding region, to form the eight example flow cells. The flow cells were subsequently dried.

One of the flow cells (Flow cell 1-2) included an acrylic or polyacrylate-based pressure-sensitive adhesive as the solvent-responsive bonding material. A second of the flow cells (Flow cell 2-2) included the solvent-responsive bonding material of Flow cell 1-2, with additional polymer crosslinker additives therein. A third of the flow cells (Flow cell 3-2) included the solvent-responsive bonding material of Flow cell 1-2, with 1.75× the polar group content of the solvent-responsive bonding material of Flow cell 1-2. A fourth of the flow cells (Flow cell 4-2) included the solvent-responsive bonding material of Flow cell 1-2, with different polymer side chains relative to the solvent-responsive bonding material of Flow cell 1-2. A fifth of the flow cells (Flow cell 5-2) included the solvent-responsive bonding material of Flow cell 1-2, with 0.5× the polar group content of the solvent-responsive bonding material of Flow cell 1-2 and different polymer side chains, relative to the solvent-responsive bonding material of Flow cell 1-2. A sixth of the flow cells (Flow cell 6-2) included the solvent-responsive bonding material of Flow cell 1-2, with 1.75× the polar group content of the solvent-responsive bonding material of Flow cell 1-2 and different polymer side chains. A seventh of the flow cells (Flow cell 7-2) included the solvent-responsive bonding material of Flow cell 1-2, with 0.125× the amount of crosslinker additive relative to the solvent-responsive bonding material of Flow cell 1-2. An eighth of the flow cells (Flow cell 8-2) included the solvent-responsive bonding material of Flow cell 1-2, with 0.5× the amount of crosslinker additive relative to the solvent-responsive bonding material of Flow cell 1-2.

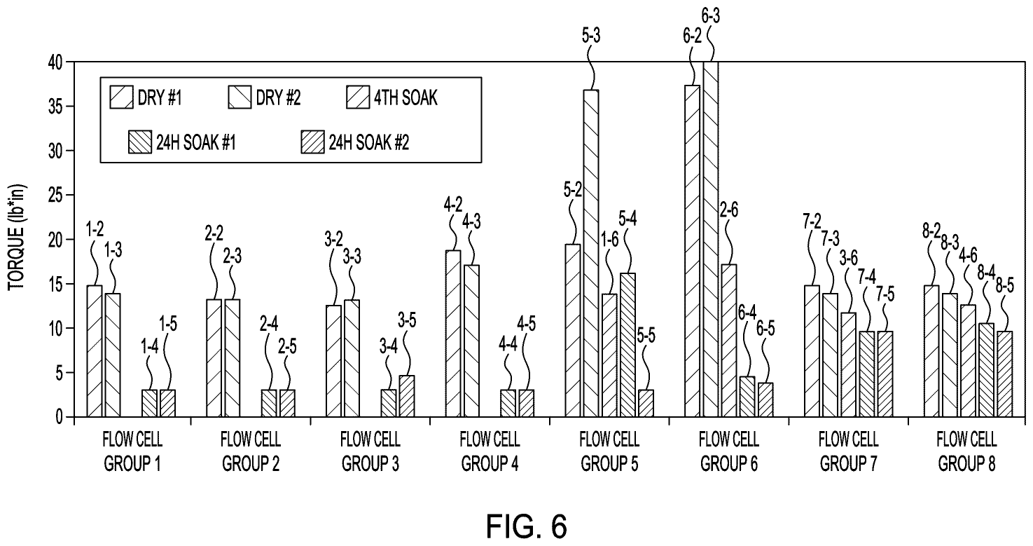

After drying, the flow cells were exposed to a shearing test. In this test, the lid was removed from the substrate using either a Milwaukee drill or a Makita drill, which was used to turn a screw that pushed a shearing blade between the respective interposers and lids of each of Flow cells 1-2 through 8-2. The Makita drill was used after the Milwaukee drill when then shearing torque required for lid removal exceeded 19/4 in-lbs. An initial shearing torque measurement (in Ib*in) was taken for each of the eight flow cells. These shearing torque measurements are shown in FIG. 6 as the “Dry #1” measurements.

This experiment was duplicated for eight additional flow cells (Flow cells 1-3 through 8-3). The results are also shown in FIG. 6 as the “Dry #2” measurements.

Eight additional flow cells (Flow cells 1-4 through 8-4) were prepared in a manner similar to that of Flow cells 1-2 to 8-2 and Flow cells 1-3 to 8-3.

Flow cells 1-4 through 4-4 were soaked in tap water for about 24 hours, and flow cells 5-4 through 8-4 were then soaked in deionized (DI) water for about 24 hours. After soaking, the lid was exposed to the shearing test. The shearing torque measurement (in Ib*in) was taken for each of the eight flow cells. These shearing torque measurements are shown in FIG. 6 as the “24 h Soak #1” measurements.

This process was repeated for eight additional example flow cells (Flow cells 1-5 through 8-5). The shearing torque measurement (in Ib*in) was taken for each of the eight flow cells. These shearing torque measurements are shown in FIG. 6 as the “24 h Soak #2” measurements.

Four additional flow cells (Flow cell 1-6 through 4-6) were prepared in a manner similar to flow cells 4-2 through 8-2. Flow cells 1-6 through 4-6 were soaked in DI water for 4 hours. The lid of each flow cell was exposed to the shearing test. The shearing torque measurement (in Ib*in) was taken for each of the eight flow cells. These shearing torque measurements are shown in FIG. 6 as the “4 h Soak” measurements.

As can be seen in FIG. 6 after being soaked in tap or DI water, the respective adhesives exhibited a significantly lower shearing torque as soak time increased. For example, flow cells 6-2 and 6-3 respectively exhibited a shearing torque of about 37 lb*in (Dry #1) and 40 lb*in (Dry #2) prior to respective soakings in DI water. In contrast, flow cells 6-4 and 6-5 each exhibited a shearing torque of about 4 lb*in after soaking in DI water for about 24 hours (24 h Soak #1 and #2). For flow cell 1-6 through 4-6, the 4-hour soak reduced the shearing torque needed to remove the lid compared to the dry examples, but the longer soaking times aided in further reducing of adhesive properties of this adhesive.

Overall, these results demonstrate that the solvent-responsive bonding materials disclosed herein may be used to detach flow cell components, such as interposers and substrates, from one another by exposing the solvent-responsive bonding materials to preselected solvents for predetermined time periods. After exposure to the preselected solvents, the shearing torque needed to remove the interposers can be significantly decreased, such that less shearing torque is generated/required when attempting to remove the interposers from the substrates, relative to the amount of shearing torque generated/required prior to solvent exposure.

### Example 3

Two additional flow cells (e.g., “Flow cell 1-7” and “Flow cell 2-7”) were prepared. Each of the substrates of the flow cells was a planar multi-layer substrate having a resin over a glass base support. A hydrogel adhesive (an acrylic or polyacrylate-based pressure-sensitive adhesive, the adhesive of Flow cell 1-2) was applied to the planar surfaces of the respective substrates and glass lids were attached through an interposer bonded to the lid.

Flow cell 1-7 was de-bonded via a shearing operation prior to any soaking, whereas Flow cell 2-7 was de-bonded via a shearing operation after being soaked in tap water for 24 hours.

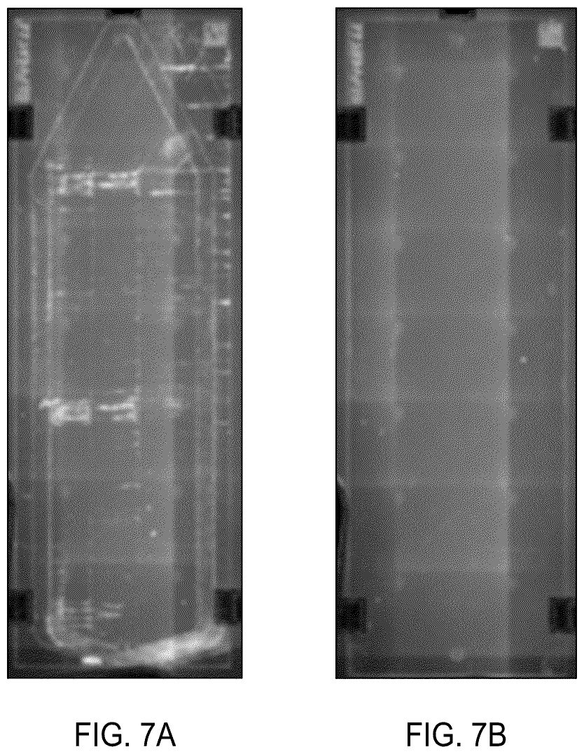

A microscopic image was taken of each of Flow cell 1-7 and Flow cell 2-7 using a NIKON™ VMA microscope, after debonding had been performed. These images are respectively reproduced in FIG. 7A and FIG. 7B. As can be seen in FIG. 7A, a significant amount of residue (i.e., of the solvent-responsive bonding material) remained on Flow cell 1-7, which had not been soaked in water. In contrast, in FIG. 7B, no residue was observed on Flow cell 2-7, which had been soaked in water for 24 hours.

These results indicate that the solvent-responsive bonding materials disclosed herein and corresponding reaction conditions (i.e., exposure to a preselected solvent) may be used to detach flow cell components from one another while leaving behind minimal amounts of residue.

### Example 4

Two flow cells were used in this example.

One flow cell (1-8) was a planar multi-layer substrate having a patterned resin over a glass base support. A non-switchable adhesive was applied to the planar surfaces of the substrate and a glass lid was attached through an interposer bonded to the lid.

The other flow cell (2-8) was a planar multi-layer substrate having a patterned resin over a glass base support. A UV switchable adhesive was applied to the planar surfaces of the substrate and a glass lid was attached through an interposer bonded to the lid.

A library based on PhiX Control v3 (a genomic library) was introduced into each of the flow cells and clustering was performed according to a sequencing by synthesis protocol. Each of the flow cells was debonded by a shearing operation.

Once debonded, mouse kidney tissue was introduced into three wells on each of the substrates, and poly A RNA was introduced into three other wells on each of the substrates. The flow cells were exposed to first strand cleavage, and second strand conversion qPCR, and the eluted sample libraries were sequenced.

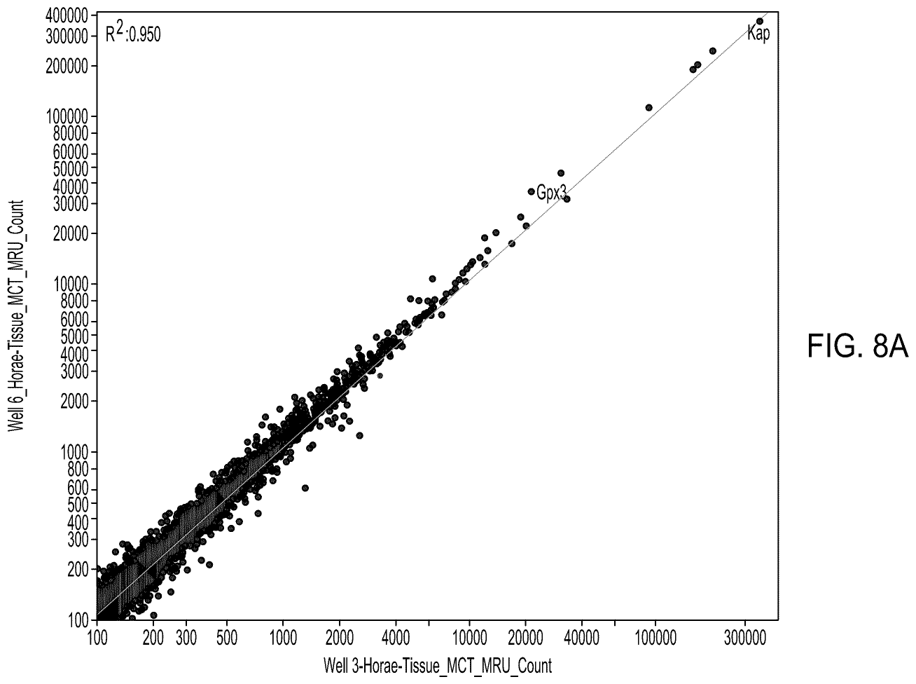

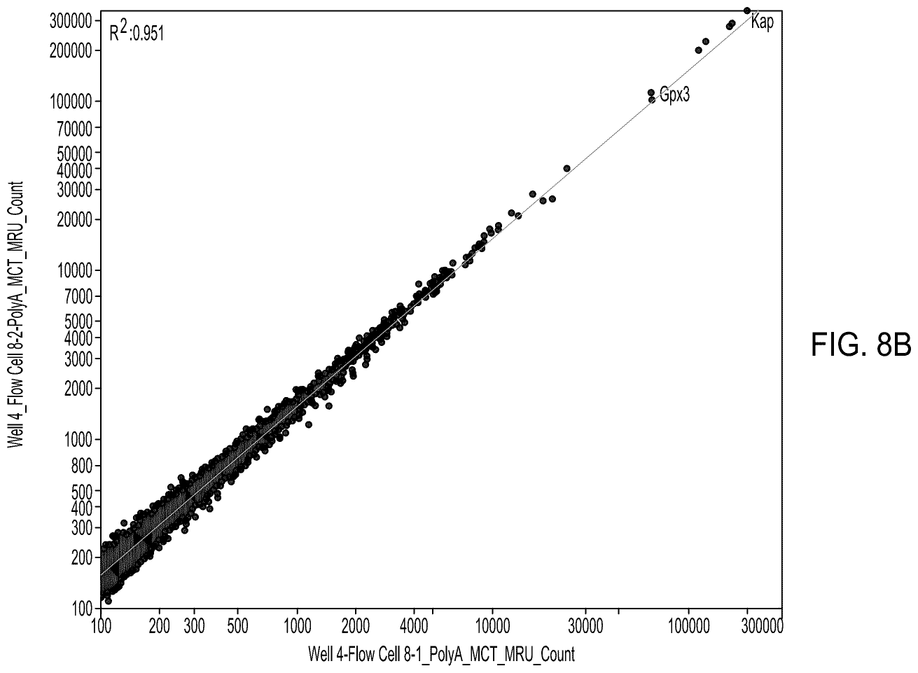

FIG. 8A illustrates the total gene coverage assessment for the mouse kidney sample in two different wells of the 2-8 flow cell. FIG. 8B illustrates the total gene coverage assessment for the poly A RNA sample in one of the wells of each of the substrates (1-8 results on the Y axis and 2-8 results on the X axis). The correlation between different wells of the same flow cell or well of the two different flow cells indicates that the debonding process described herein does not deleteriously affect the downstream clustering or sequencing, as indicated by the statistical data that was obtained and that is depicted in FIG. 8A and FIG. 8B.

### ADDITIONAL NOTES

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

While several examples have been described in detail, it is to be understood that the disclosed examples may be modified. Therefore, the foregoing description is to be considered non-limiting.

1. A flow cell, comprising:
a first substrate including an active region and a bonding region that is spatially separate from the active region;
a polymeric hydrogel positioned at the active region;
an interposer having a first side that is attached to the bonding region and having a second side that is opposed to the first side;
a light-switchable bonding material in contact with at least one of the first side or the second side; and
a lid or a second substrate attached to the second side of the interposer.

2. The flow cell as defined in claim 1, further comprising a non-switchable bonding material in contact with the second side, wherein the non-switchable bonding material attaches the second side to the lid or to the second substrate, and wherein the light-switchable bonding material is in contact with the first side and attaches the first side to the bonding region.

3. (canceled)

4. The flow cell as defined in claim 1, further comprising a non-switchable bonding material in contact with the first side, wherein the non-switchable bonding material attaches the first side to the bonding region, and wherein the light-switchable bonding material is in contact with the second side and attaches the second side to the lid or to the second substrate.

5. (canceled)

6. The flow cell as defined in claim 1, wherein the light-switchable bonding material:
is in contact with both the first side and the second side;
attaches the first side to the bonding region; and
attaches the second side to the lid or to the second substrate.

7. The flow cell as defined in claim 1, wherein the light-switchable bonding material is selected from the group consisting of an epoxy acrylate, an acrylic polymer, silicone, a cyanoacrylate, a base polymer combined with an oligomer and a photoinitiator, an acrylic copolymer mixed with a multifunctional monomer and a photo-initiator, and a combination thereof.

8. The flow cell as defined in claim 1, further comprising a plurality of primers attached to the polymeric hydrogel.

9. The flow cell as defined in claim 1, wherein the second substrate is attached to the second side, and wherein the second substrate includes:
a second active region that is spatially separate from a second bonding region; and
the polymeric hydrogel positioned at the second active region.

10. The flow cell as defined in claim 1, wherein the bonding region includes a plurality of depressions defined in the first substrate.

11. (canceled)

12. A method of using the flow cell of claim 1, the method comprising:
exposing the light-switchable bonding material to a predetermined wavelength of light, thereby reducing adhesive properties of the light-switchable bonding material; and
separating the first substrate from the lid or from the second substrate.

13. The method as defined in claim 12, wherein the separating is performed at:
an interface between the first side and the bonding region when the light-switchable bonding material is in contact with the first side; or
an interface between the second side and the lid or second substrate when the light-switchable bonding material is in contact with the second side.

14. The method as defined in claim 13, wherein the separating of the first substrate from the lid or from the second substrate involves passing a taut layer of metal foil having a width ranging from about 1 mm to about 50 mm through:
the interface between the first side and the bonding region; or
the interface between the second side and the lid or second substrate.

15.-28. (canceled)

29. A method of making a flow cell, comprising:
applying a polymeric hydrogel to a first region of a first substrate, thereby forming an active region that is spatially separate from a bonding region of the first substrate;
applying a light-switchable bonding material to i) the bonding region or ii) at least one of a first side or a second side of an interposer, wherein the first side is opposed to the second side;
attaching the first side of the interposer to the bonding region; and
attaching a lid or a second substrate to the second side of the interposer.

30. The method as defined in claim 29, wherein the light-switchable bonding material:
is applied to the bonding region or to the first side of the interposer; and
attaches the first side of the interposer to the bonding region;
and wherein attaching the lid or the second substrate to the second side of the interposer involves:
applying a non-switchable bonding material to the second side; and
contacting the lid or the second substrate with the non-switchable bonding material.

31. (canceled)

32. The method as defined in claim 29, wherein prior to attaching the first side of the interposer to the bonding region, the method further comprises forming a plurality of depressions in the bonding region, and wherein after attaching the first side of the interposer to the bonding region, the light-switchable bonding material at least partially fills the plurality of depressions in the bonding region.

33. The method as defined in claim 29, wherein the light-switchable bonding material:
is applied to the second side of the interposer; and
attaches the second side of the interposer to the lid or to the second substrate; and wherein attaching the first side of the interposer to the bonding region involves:
applying a non-switchable bonding material to the first side or the bonding region; and
then placing the first side and the bonding region in contact with each other.

34. (canceled)

35. The method as defined in claim 29, wherein the light-switchable bonding material:
is applied to both the first side and the second side of the interposer;
attaches the first side of the interposer to the bonding region; and
attaches the second side of the interposer to the lid or to the second substrate.

36. (canceled)

37. The method as defined in claim 29, further comprising attaching a plurality of primers to the polymeric hydrogel.

38. The method as defined in claim 29, wherein the second substrate is attached to the second side of the interposer, and wherein the second substrate includes:
a second active region that is spatially separate from a second bonding region; and
the polymeric hydrogel positioned at the second active region.

39. The method as defined in claim 29, wherein:
the light-switchable bonding material exhibits improved adhesion when exposed to ultraviolet light and exhibits reduced adhesion when exposed to visible light; and
the method further comprises:
placing the first side and the bonding region in contact with each other after the light-switchable bonding material is applied; and
then exposing the light-switchable bonding material to ultraviolet light.

40.-50. (canceled)

51. A biological sequencing kit, comprising:
a flow cell including:
a first substrate having an active region and a bonding region that is spatially separate from the active region;
a polymeric hydrogel positioned at the active region;
an interposer having a first side that is attached to the bonding region and having a second side that is opposed to the first side;
a light-switchable bonding material in contact with at least one of the first side or the second side, the light-switchable bonding material exhibiting a decrease in adhesive properties upon exposure to visible light; and
a lid or a second substrate attached to the second side of the interposer; and
a visible light blocking storage container housing the flow cell.

52.-54. (canceled)


SEQUENCE LISTING (13 sequences)

SEQ ID NO: 1 - DNA, 29 bp, synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, Synthetic construct
  source 1..29: mol_type = other DNA
  modified_base 23: mod_base = i
aatgatacggcgaccaccgaganctacac

SEQ ID NO: 3 - DNA, 29 bp, Synthetic construct
  source 1..29: mol_type = other DNA
  modified_base 23: mod_base = OTHER; note = alkene-thymidine
aatgatacggcgaccaccgaganctacac

SEQ ID NO: 4 - DNA, 24 bp, Synthetic construct
  source 1..24: mol_type = other DNA
  modified_base 22: mod_base = OTHER; note = 8-oxoguanine
caagcagaagacggcatacganat

SEQ ID NO: 5 - DNA, 24 bp, Synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, Synthetic construct
  modified_base 20: mod_base = OTHER; note = 8-oxoguanine
  modified_base 22: mod_base = OTHER; note = 8-oxoguanine
  source 1..24: mol_type = other DNA
caagcagaagacggcatacnanat

SEQ ID NO: 7 - DNA, 24 bp, Synthetic construct
  source 1..24: mol_type = other DNA
  modified_base 22: mod_base = OTHER; note = uracil
caagcagaagacggcatacgatat

SEQ ID NO: 8 - DNA, 24 bp, Synthetic construct
  source 1..24: mol_type = other DNA
  modified_base 20: mod_base = OTHER; note = uracil
caagcagaagacggcatactagat

SEQ ID NO: 9 - DNA, 29 bp, Synthetic construct
  modified_base 23: mod_base = OTHER; note = a thymine nucleotide analog having an allyl functionality
  source 1..29: mol_type = other DNA
aatgatacggcgaccaccgaganctacac

SEQ ID NO: 10 - DNA, 35 bp, Synthetic construct
  source 1..35: mol_type = other DNA
gctggcacgtccgaacgcttcgttaatccgttgag

SEQ ID NO: 11 - DNA, 35 bp, Synthetic construct
  source 1..35: mol_type = other DNA
cgtcgtctgccatggcgcttcggtggatatgaact

SEQ ID NO: 12 - DNA, 35 bp, Synthetic construct
  source 1..35: mol_type = other DNA
acggccgctaatatcaacgcgtcgaatccgcaact

SEQ ID NO: 13 - DNA, 34 bp, Synthetic construct
  source 1..34: mol_type = other DNA
gccgcgttacgttagccggactattcgatgcagc